SEQUENCE LISTING (7 sequences)

SEQ ID NO: 1 - protein, 107 aa, Unknown
  Synthetic
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIK

SEQ ID NO: 2 - protein, 116 aa, Unknown
  Synthetic
EVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSS

SEQ ID NO: 3 - protein, 112 aa, Unknown
  Synthetic
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIK

SEQ ID NO: 4 - protein, 113 aa, Unknown
  Synthetic
QVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSS

SEQ ID NO: 5 - protein, 502 aa, Unknown
  Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAPSSSPMEDIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSGGCGGGEVAALEKEVAALEKEVAALEKEVAALEKGGGNS

SEQ ID NO: 6 - protein, 270 aa, Unknown
  Synthetic
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIKGGGSGGGGEVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSSGGCGGGKVAALKEKVAALKEKVAALKEKVAALKE

SEQ ID NO: 7 - protein, 227 aa, Unknown
  Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

(19) United States
(12) Patent Application Publication
(10) Pub. No.: US 2021/0032333 A1
(43) Pub. Date: Feb. 4, 2021

(54) Methods and Compositions for Reducing Immunogenicity By Non-Depletional B Cell Inhibitors

(71) Applicant: Provention Bio, Inc., Lebanon, NJ (US)

(72) Inventors: Francisco Leon, Lebanon, NJ (US); Paul Dunford, Lebanon, NJ (US)

(21) Appl. No.: 16/943,849

(22) Filed: Jul. 30, 2020

Related U.S. Application Data
(60) Provisional application No. 62/880,240, filed on Jul. 30, 2019.

(51) Int. Cl. C07K 16/28; A61K 48/00; A61K 39/395; A61K 31/436; A61K 38/48; A61P 37/02

(52) U.S. Cl. CPC C07K 16/2803; A61K 48/00; C07K 16/283; A61K 39/3955; C07K 2317/52; A61K 38/4873; A61P 37/02; C07K 2317/31; A61K 31/436

(57) ABSTRACT

Disclosed herein, in one aspect, is a method of reducing immunogenicity, comprising administering to a patient receiving or having received a biological therapeutic agent, an effective amount of B cell inhibitor that is non-depletional. Related compositions are also provided.

### CROSS REFERENCE TO RELATED APPLICATIONS

This application claims priority to and the benefit of U.S. Provisional Application No. 62/880,240 filed Jul. 30, 2019, incorporated herein by reference in its entirety.

### SEQUENCE LISTING

The ASCII text file submitted on Jul. 30, 2020 via EFS-Web, entitled “010801seq.txt” created on Jul. 30, 2020, having a size of 13,266 bytes, is incorporated herein by reference in its entirety.

### FIELD

The present disclosure generally relates to compositions and methods for reducing immunogenicity of biological therapeutics, and more particularly to do so by means of B cell inhibitors which are not depletional.

### BACKGROUND

The use of biologics such as antibodies and polypeptides as therapeutics has the associated risk of generating undesirable immune responses in patients, typically defined by the generation of anti-drug antibody (ADA) responses. Such responses can be motivated by the presence of “foreign” epitopes in the molecule and can be exacerbated by extrinsic factors, such as the genomic and disease background of the patient, the dosing and administration regime utilized, the formulation, and the route of administration and impurities, amongst others. These immune responses can have a variety of consequences, from altered pharmacology, to increased drug clearance or neutralization and loss of therapeutic efficacy. In extreme cases, protein therapeutics can cause the development of severe allergic and anaphylactic reactions, with considerable risk to the patient.

Another well-characterized immune reaction to “foreign” agents is the so-called graft or transplant rejection (also termed host-versus-graft reaction), in which the endogenous immune system reacts against, causing the destruction of, foreign tissue. Tissue rejection can be mediated by humoral and cellular immune responses. In the case of genetically modified cells generated for the purpose of incorporating a missing copy of a gene (gene therapy) or to help the patient eliminating cancerous cells (e.g., CAR-T therapies), there is a risk that some of the “machinery” utilized for the genetic modification of the cells could be “presented” by the modified cells and be recognized by the host as a “foreign” agent. Such recognition would trigger a rejection reaction, which could potentially render ineffective such treatments or, in severe cases, potentially cause auto-immune reactions.

More recently, the advent of genetic therapies has seen the substantial obstacle of immunogenicity of the viral vector utilized to administer the transgene, as well as the immunogenicity of the transgene protein itself after expression by the recipient's cells. The immunogenicity of vectors and transgenes results in: 1) diminished efficacy as vector and transgene they are bound and cleared by the antibodies generated by the recipient; 2) need for increased doses, which increase safety risks and costs; 3) difficulty or impossibility to re-dose if the subject develops antibodies against the vector or transgene after a prior dose. Sometimes, the recipients have pre-existing antibodies against the vector even before the first administration, die to cross-reaction with naturally-occurring viruses. Other therapies based on viruses (e.g., oncolytic viruses in cancer) and gene editing therapies (e.g., CRISPR-Cas9-based therapies), also suffer from immunogenicity.

, As such, a need exists for methods and compositions for reducing immunogenicity induced by various biological therapeutics, including without limitation, antibodies, cell therapy, and gene therapy.

### SUMMARY

Disclosed herein, in one aspect, is a method of reducing immunogenicity, comprising administering to a patient receiving or having received a biological therapeutic agent, an effective amount of B cell inhibitor that is non-depletional.

In some embodiments, the biological therapeutic agent is selected from one or more of: gene therapy, gene editing therapy, messenger RNA (mRNA) therapy, oncolytic viruses, enzyme replacement therapy, antibody therapy, protein therapeutics, and cell therapy. In some embodiments, the biological therapeutic agent is gene therapy.

In some embodiments, the B cell inhibitor is a CD32B×CD79B bi-specific antibody capable of immunospecifically binding an epitope of CD32B and an epitope of CD79B. In some embodiments, the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

In some embodiments, the CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

In some embodiments, the method can further include administering the Fc diabody at a dose of between about 5 mg/kg and about 40 mg/kg, and at a dosage regimen of between one dose per 2 week and one dose per 6 weeks. In some embodiments, the method can include administering the Fc diabody at a dose of about 10 mg/kg, and at a dosage regimen of one dose per 4 weeks. In some embodiments, the method can include administering 3 doses of the Fc diabody at a dose of about 10 mg/kg at 2-6 week intervals.

In some embodiments, the method can include administering a first dose about 2-6 weeks (e.g., 4 weeks) prior to administration of the biological therapeutic agent, a second dose at about the same time as administration of the biological therapeutic agent, and a third dose about 2-6 weeks (e.g., 4 weeks) after administration of the biological therapeutic agent.

In some embodiments, the Fc diabody results in inhibition of its own immunogenicity upon administration, with lower prevalence and/or titers of anti-drug antibodies (ADA) at increased doses. In some embodiments, the ADA does not neutralize the Fc diabody.

In some embodiments, the Fc diabody, in a dose-dependent fashion, binds to at least 80% B cells upon administration, and remains bound to at least 50% of the B cells for at least 4 weeks after last administration.

In some embodiments, the Fc diabody results in sustained inhibition of immunoglobulin production without depleting circulating B cells. In some embodiments, the immunoglobulins include one or more of IgM, IgA, IgG and IgE.

In some embodiments, the method can further include monitoring the patient by examining the presence of specific antibodies against the biological therapeutic agent. In some embodiments, the method can further include administering one or more dose of the B cell inhibitor to further modulate immunogenicity.

In some embodiments, the method can further include co-administering one or more immune-modulators, such as sirolimus, rapamycin, abatacept, teplizumab and immunoglobulin G-degrading enzyme of Streptococcus pyogenes.

Also provided herein are pharmaceutical compositions comprising the non-depletional B cell inhibitors disclosed herein, provided (e.g., packaged) at therapeutically effective unit doses. Instructions for dosage regimens as disclosed herein can also be provided.

### BRIEF DESCRIPTION OF THE DRAWINGS

FIG. 1: Schematic Overview of the Study.

FIGS. 2A-2C: Mean (±SD) PRV-3279 Serum Concentrations (ng/mL) versus Time by Day on Linear Scale (Pharmacokinetic Population) (FIG. 2A: Day 1, FIG. 2B: Day 15, FIG. 2C: Day 29).

FIGS. 3A-3C: Mean PRV-3279 Serum Concentrations (ng/mL) versus Time by Day on Semi-logarithmic Scale (Pharmacokinetic Population) (FIG. 3A: Day 1, FIG. 3B: Day 15, FIG. 3C: Day 29).

FIGS. 4A-4B: Mean (±SD) of PRV-3279 Serum Concentrations (ng/mL) versus Time (Day) by ADA Result by Dose (Pharmacokinetic Population) (FIG. 4A: 3 mg/kg, FIG. 4B: 10 mg/kg).

FIG. 5: Box Plot of PRV-3279 Serum Pharmacokinetic Parameter by ADA Result by Dose (Pharmacokinetic Population).

FIG. 6: Arithmetic Mean (±SEM) of % MaxBinding of % anti-E/K+(CD3−/CD19+) by Time and Treatment (Pharmacodynamic Population).

FIG. 7: Arithmetic Mean (±SEM) of Cell Numbers of B Cells (CD19+)−(cells/returned to μL) (Pharmacodynamic Population).

FIG. 8: Arithmetic Mean (±SEM) of Reduction in Circulating Serum IgM Levels (Safety Population).

FIG. 9: Arithmetic Mean (±SEM) of Reduction in Circulating Serum IgE Levels (Safety Population).

FIG. 10: Arithmetic Mean (±SEM) of Reduction in Circulating Serum IgG Levels (Safety Population).

### DETAILED DESCRIPTION

Disclosed herein, in one aspect, is a method of reducing immunogenicity, comprising administering to a patient receiving or having received a biological therapeutic agent, an effective amount of B cell inhibitor that is non-depletional. In some embodiments, the B cell inhibitor is a CD32B×CD79B bi-specific antibody such as those disclosed in U.S. Publication No. 2016/0194396, WIPO Publication Nos. WO 2015/021089 and WO2017/214096, each incorporated by reference in its entirety.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The term “substantially” means more than 50%, preferably more than 80%, and most preferably more than 90% or 95%.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, host cells, and/or vectors of the invention can be used to achieve methods and proteins of the invention.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

A “nucleic acid,” “nucleic acid molecule,” “oligonucleotide” or “polynucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. In some embodiments, the invention is directed to a polynucleotide encoding any one of the polypeptides disclosed herein, e.g., is directed to a polynucleotide encoding a Cas protein or variant thereof. In some embodiments, the invention is directed to a polynucleotide encoding Cas3, Cas9, Cas1O or variants thereof.

A “gene” refers to an assembly of nucleotides that encode a polypeptide, and includes cDNA and genomic DNA nucleic acid molecules. “Gene” also refers to a nucleic acid fragment that can act as a regulatory sequences preceding (5′ non-coding sequences) and following (3′ non-coding sequences) the coding sequence.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

Throughout the present specification, the numbering of the residues in the constant region of an IgG Heavy Chain is that of the EU index as in Kabat et al., Sequences of Proteins of Immunological Interest, 5th Ed. Public Health Service, NH1, MD (1991) (“Kabat”), expressly incorporated herein by references. The term “EU index as in Kabat” refers to the numbering of the human IgG1 EU antibody. Amino acids from the Variable Domains of the mature heavy and Light Chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,”. J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule.

The terms “bispecific antibody molecule,” “diabody” and “Dual Affinity Re-Targeting (DART®)” antibody are used interchangeably herein and refer to an antibody molecule that has specificity for more than one (e.g., two, three, four, or more) epitope and/or antigen. In some embodiments, the antibody can be diabodies or scaffolds capable of antigen binding, such as those disclosed in U.S. Publication No. 2016/0194396, WIPO Publication Nos. WO 2015/021089 and WO2017/214096, each incorporated by reference in its entirety. In some embodiments, the antibody can be CD32B×CD79B bispecific diabodies (i.e., “CD32B×CD79B diabodies,” and such diabodies that additionally comprise an Fc domain (i.e., “CD32B×CD79B Fc diabodies”). In one embodiment, the antibody can be a humanized CD32B×CD79B DART® antibody, produced in Chinese hamster ovary cells with a molecular weight of 111.5 kDa.

“Antigen” (Ag) as used herein refers to a macromolecule, including all proteins or peptides. In some embodiments, an antigen is a molecule that can provoke an immune response, e.g., involving activation of certain immune cells and/or antibody generation. Antigens are not only involved in antibody generation. T cell receptors also recognized antigens (albeit antigens whose peptides or peptide fragments are complexed with an MEW molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chotia control so that the frameworks have consistent numbering across antibodies.

In some embodiments, the antigen-binding fragment of an antibody (e.g., when included as part of a fusion molecule) can lack or be free of a full Fc domain. In certain embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

As used herein, “administering” and similar terms mean delivering the composition to an individual being treated. Preferably, the compositions of the present disclosure are administered by, e.g., parenteral, including subcutaneous, intramuscular, or preferably intravenous routes.

As used herein, an “effective amount” means the amount of bioactive agent or diagnostic agent that is sufficient to provide the desired local or systemic effect at a reasonable risk/benefit ratio as would attend any medical treatment or diagnostic test. This will vary depending on the patient, the disease, the treatment being effected, and the nature of the agent. A therapeutically effective amount will vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg of an antibody or antigen binding portion thereof, as provided herein. Dosing may be, e.g., every week, every 2 weeks, every three weeks, every 4 weeks, every 5 weeks or every 6 weeks. Dosage regimens may be adjusted to provide the optimum therapeutic response. An effective amount is also one in which any toxic or detrimental effects (side effects) of the agent are minimized and/or outweighed by the beneficial effects. Administration may be intravenous at exactly or about 6 mg/kg or 12 mg/kg weekly, or 12 mg/kg or 24 mg/kg biweekly. Additional dosing regimens are described below.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions etc.

The term “immunogenicity” refers to the ability of a particular substance, such as an antigen or epitope, to provoke an immune response, which can be humoral and/or cell-mediated, in the body of a human and other animal. In some embodiments, administration of the composition of the present disclosure reduces the immunogenicity of, and/or increases the immune tolerance to, a biological substance such as therapeutics. “Tolerance” or “immune tolerant” as used herein, refers to the absence of an immune response to a specific antigen (e.g., the therapeutic biologic) in the setting of an otherwise substantially normal immune system.

A “major histocompatibility complex” or “MHC” protein as used herein refers to a set of cell surface molecules encoded by a large gene family that play a significant role in the immune system of vertebrates. A key function of these proteins is to bind peptide fragments derived from endogenous or exogenous (foreign) proteins and display them on the cell surface for recognition by the appropriate T-cells of the host organism. The MEW gene family is divided into three subgroups: Class I, Class II, and Class III. The human MEW Class I and Class II genes are also referred to as human leukocyte antigen (HLA)—HLA Class I and HLA Class II, respectively. Some of the most studied HLA genes in humans are the nine MHC genes: HLA-A, HLA-B, HLA-C, HLA-DPA1, HLA-DPB1, HLA-DQA1, HLA-DQB1, HLA-DRA, HLA-DRB1 and HLA-DRB345.

Various aspects of the disclosure are described in further detail below. Additional definitions are set out throughout the specification.

### Non-Depleting B Cell Inhibitors and Pharmaceutical Compositions

In various embodiments, a B cell inhibitor can be used to reduce or modulate immunogenicity. In some embodiments, such B cell inhibitors are non-depletional immunomodulators. As used herein, “non-depletional” or “non-depleting” means that the inhibitor or immunomodulator does not completely deplete B cell activities. On the other hand, “depletion” of B cells means that the agent acts to eliminate or destroy B cells, such as anti-CD20 antibodies, e.g., Rituximab. Thus, in one embodiment, the non-depletional B cell inhibitors or immunomodulators disclosed herein are not Rituximab. In some embodiments, the non-depletional B cell inhibitors or immunomodulators are not anti-CD20 antibodies or other CD20 inhibitors.

Exemplary non-depletional B cell inhibitors include, but are not limited to, CD32B×CD79B bi-specific inhibitors; CD32B modulators; B cell receptor (BCR) blockers, e.g., anti-CD22 molecules; B cell survival and activation inhibitors, e.g., B-cell activating factor (BAFF) or A proliferation-inducing ligand (APRIL) inhibitors such as belimumanb; anti-CD40 and anti-CD40L molecules; and Bruton's tyrosine kinase (BTK) inhibitors such as Ibrutinib (PCI-32765) and Acalabrutinib.

In some embodiments, the B cell inhibitor can be a CD32B×CD79B bi-specific antibody such as those disclosed in U.S. Publication No. 2016/0194396, WIPO Publication Nos. WO 2015/021089, and WO2017/214096, all incorporated by reference in its entirety, or an antigen-binding fragment thereof.

An exemplary CD32B×CD79B bispecific diabody can comprise two or more polypeptide chains, and can comprise:
(1) a VL Domain of an antibody that binds CD32B (VL_{CD32B}), such VL_{CD32B} Domain having the sequence (SEQ ID NO: 1):

DIQMTQSPSS LSASVGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIK

(2) A VH Domain of an antibody that binds CD32B (VH_{CD32B}), such VH_{CD32B} Domain having the sequence (SEQ ID NO: 2):

EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSS

(3) A VL Domain of an antibody that binds CD79B (VL_{CD79B}), such VL_{CD79B} Domain having the sequence (SEQ ID NO: 3):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IK

(4) A VH Domain of an antibody that binds CD79B (VH_{CD79B}), such VH_{CD79B} Domain having the sequence (SEQ ID NO: 4):

QVQLVQSGAE VKKPGASVKV SCKASGYTFT SYWMNWVRQA
PGQGLEWIGM IDPSDSETHY NQKFKDRVTM TTDTSTSTAY
MELRSLRSDD TAVYYCARAM GYWGQGTTVT VSS

In one embodiment, the B cell inhibitor can be PRV-3279, a humanized CD32B×CD79B Dual Affinity Re-Targeting (DART®) protein produced in Chinese hamster ovary cells with a molecular weight of 111.5 kDa. DART® proteins are bispecific, antibody-based molecules that can bind 2 distinct antigens simultaneously. PRV-3279 is designed to target CD32B (Fc gamma receptor IIb) and CD79B (immunoglobulin-associated beta subunit of the B cell receptor (BCR) complex) on B lymphocytes. Co-ligation of CD32B and CD79B in preferential cis-binding mode on B lymphocytes triggers CD32B-coupled immunoreceptor tyrosine-based inhibitory motif signaling, which decreases antigen-mediated naïve and memory B cell activation without broad depletion. To prolong in vivo half-life, PRV-3279 also contains a human immunoglobulin G (IgG)1 Fc region that has been mutated to greatly reduce or eliminate undesired binding to FcγRs and complement but retains affinity for the neonatal FcR binding to take advantage of the IgG salvage pathway mediated by this receptor.

The CD32B molecule is a transmembrane inhibitory receptor expressed widely on B cells and other immune effector cells such as macrophages, neutrophils, and mast cells. The anti-CD32B component of PRV-3279 is based on a humanized version of MacroGenics' proprietary murine monoclonal antibody (mAb) 8B5. CD79B is an essential signal transduction component of the BCR that is expressed exclusively on B cells. The anti-CD79B component of PRV-3279 is based on a humanized version of the murine mAb CB3.

In one embodiment, PRV-3279 comprises the following sequence (the CDRs are underlined and coil domains are in bold):

Chain1 (Fc-CD32BVL-CD79bVH-E coil):
(SEQ ID NO.: 5)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLWCLVK
GFYPSDIAVEWESNGQPENNYKTTPPVLDSDGSFFLYSKLTVDKSRWQQG
NVFSCSVMHEALHNHYTQKSLSLSPGKAPSSSPMEDIQMTQSPSSLSASV
GDRVTITCRASQEISGYLSWLQQKPGKAPRRLIYAASTLDSGVPSRFSGS
ESGTEFTLTISSLQPEDFATYYCLQYFSYPLTFGGGTKVEIKGGGSGGGG
QVQLVQSGAEVKKPGASVKVSCKASGYTFTSYWMNAWRQAPGQGLEWIGM
IDPSDSETHYNQKFKDRVTMTTDTSTSTAYMELRSLRSDDTAVYYCARAM
GYWGQGTTVTVSSGGCGGGEVAALEKEVAALEKEVAALEKEVAALEKGGG
NS
Chain2 (CD79bVL-CD32BVH-K coil):
(SEQ ID NO.: 6)
DVVMTQSPLSLPVTLGQPASISCKSSQSLLDSDGKTYLNWFQQRPGQSPN
RLIYLVSKLDSGVPDRFSGSGSGTDFTLKISRVEAEDVGVYYCWQGTHFP
LTFGGGTKLEIKGGGSGGGGEVQLVESGGGLVQPGGSLRLSCAASGFTFS
DAWMDWVRQAPGKGLEWVAEIRNKAKNHATYYAESVIGRFTISRDDAKNS
LYLQMNSLRAEDTAVYYCGALGLDYWGQGTLVTVSSGGCGGGKVAALKEK
VAALKEKVAALKEKVAALKE
Chain3 (Fc):
(SEQ ID NO.: 7)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLSCAVK
GFYPSDIAVEWESNGQPENNYKTTPPVLDSDGSFFLVSKLTVDKSRWQQG
NVFSCSVMHEALHNRYTQKSLSLSPGK

In another aspect, pharmaceutical compositions are provided that can be used in the methods disclosed herein, i.e., pharmaceutical compositions for reducing or suppressing immunogenicity in a subject in need thereof, e.g., while or after receiving a biologic agent that causes significant immunogenicity, or because the subject had pre-existing immunogenicity to the biotherapeutic (e.g., in the case of pre-existing anti-AAV antibodies due to prior wild-type adenoviral infections, or due to prior exposure to rAAV therapy). In some embodiments, the compositions disclosed herein can be administered to a patient before receiving a biologic agent such as antibody or gene therapy so as to prevent immunogenicity and/or reduce pre-existing antibodies.

In some embodiments, the pharmaceutical composition comprises a B cell inhibitor as disclosed herein and a pharmaceutically acceptable carrier. The B cell inhibitor can be formulated with the pharmaceutically acceptable carrier into a pharmaceutical composition. Additionally, the pharmaceutical composition can include, for example, instructions for use of the composition for the treatment of patients to reduce or suppress immunogenicity in a subject in need thereof, e.g., while or after receiving a biologic agent that causes significant immunogenicity.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg, and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks), once a month, once every 5 weeks, and once every 6 weeks.

In some embodiments, an about 5-40 mg/kg, about 5-20 mg/kg or about 10 mg/kg per dose of PRV-3279 can be administered once every 2 weeks, once every 3 weeks, once every 4 weeks, once every 5 weeks 5 or once every 6 weeks. One or more doses can be administered, such as 1 dose, 2 doses or 3 doses. Administration can be via IV infusion. Any combination of the foregoing (e.g., 3 doses of 10 mb/kg per dose, once every 4 weeks) can be used for the reduction of the immunogenicity of biotherapeutics including gene therapy products. In some embodiments, the first dose can be given 2-6 weeks (e.g., 4 weeks) before gene therapy, the second dose at around the same time of the gene therapy, and the third dose 2-6 weeks (e.g., 4 weeks) after gene therapy. Thereafter, the patient can be monitored by examining the amount of specific antibodies against gene therapy vector (e.g., rAAV) and/or the transgene. If no or little antibody can be detected, then there will be no need for additional PRV-3279. If significant amount of antibody is present, then one or more dose of PRV-3279 can be administered to further modulate immunogenicity.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (e.g., 0.005 to 70%, e.g., 0.01 to 30%) of active ingredient in combination with a pharmaceutically acceptable carrier.

### Therapeutic Uses and Methods

The compositions disclosed herein can be used to reduce or suppress immunogenicity caused by various biologic products such as gene therapy delivered by various means (e.g., AAV and other wild-type and recombinant vectors, lentivirus modified human stem cells), including the encoded transgene protein; gene editing therapies (e.g., CRISPR/Cas9); messenger RNA (mRNA) therapy (e.g., mRNA vaccines); oncolytic viruses (e.g., VSV, HSV-1); enzyme replacement therapy (e.g., Factor VIII/IX replacement); antibody- and fusion protein-based therapeutics (e.g., anti-TNF biologics); cell therapy (e.g., CAR-T therapy).

In some embodiments, the B cell immunomodulators disclosed herein can be used to improve multiple existing or emerging platforms of gene and cell based therapy, such as:
1. rAAV (recombinant adeno associated virus) vector based therapies including:
   rAAV for “traditional” viral delivery of transgene, e.g., for inherited enzyme deficiencies
   rAAV for in vivo delivery of gene editing technology (e.g., clustered regularly interspaced short palindromic repeat (CRISPR)-associated nuclease Cas9 (“CRISPR/Cas9”)
   rAAV for delivery of vaccine antibodies (e.g., Influenza)
2. Human stem cell (HSC) therapy with Lentivirus modified HSC;
3. Cas9 protein delivery (Cas9 is bacterial derived and immunogenic); and
4. Oncolytic virus such as vesicular stomatitis virus (VSV) and herpes simplex virus type 1 (HSV-1).

In some embodiments, the B cell immunomodulators disclosed herein can be used to modulate a limiting immune response elicited by multiple routes of delivery (even in sites of perceived immune privilege), such as systemic, intra-muscular, ocular (requiring high local dose results in local immune response), and central nervous system (CNS) (where leakage of viral capsid from CNS induces a systemic response that diminishes AAV uptake in CNS).

In some embodiments, the B cell immunomodulators disclosed herein can be used to modulate multiple limiting immunological pathways that are B cell dependent, including:
Development of neutralizing antibodies (nAb)
Antibody dependent cell-mediated cytotoxicity
Antibody dependent Complement mediated cytotoxicity
Autonomous B-cell Activation, e.g., via Toll-like receptors (TLR)

In some embodiments, the B cell immunomodulators disclosed herein can be used to improve multiple AAV clinical applications through B cell modulation, such as repeat dosing and/or increased AAV dose.

In some embodiments, after administration of PRV-3279, the peak plasma concentrations occurred at the end of infusion of the bispecific molecule, and there was minimal accumulation upon multiple dosing. This shows that PRV-3279 has good pharmacokinetics properties.

In some embodiments, administration of the PRV-3279 bispecific agent can result in inhibition of its own immunogenicity, i.e., lower prevalence and/or titers of anti-drug antibodies (ADA) with increased doses of the drug. This is in contrast to other immune-modulators. In addition, this suggests that increased dose of PRV-3279 such as 20 mg/kg, 30 mg/kg or 40 mg/kg can be well tolerated without added immunogenicity.

In some embodiments, it has been observed that PRV-3279 ADA does not affect pharmacokinetics (PK), pharmacodynamics (PD), safety or efficacy. This is surprising because ADA usually affects at least PK and PD. Without being bound by theory, it has been hypothesized that ADA does not neutralize PRV-3279.

In some embodiments, the PRV-3279 bispecific agent, in a dose-dependent fashion, binds to most (e.g., >80-90%) B cells, including both naïve and memory phenotypes, upon administration, and remains bound to at least 50% of the B cells for at least 4 weeks after last administration of certain higher dosages of the drug. This shows sustained durability of the PD effect of PRV-3279, and supports once every month (or longer) administration.

In some embodiments, the dose dependency and sustained B cell binding by the PRV-3279 bispecific drug leads to durable inhibition of immunoglobulin production in the absence of depletion of any circulating cell subset, including B cells. Immunoglobulins reduced in peripheral blood include IgM, IgA, IgG and IgE. The inhibition can be observed in the absence or presence (e.g., vaccination) of antigen stimulation. This is an advantageous safety feature of PRV-3279 as a non-depleting agent, so that the patient can retain the circulating cells such as B cells to function as part of the immune system. In contrast, patients receiving depleting agents (e.g., rituximab, ocrelizumab, inebilizumab) take a long time to recover (e.g., a year).

### EXAMPLES

The following examples, including the experiments conducted and results achieved, are provided for illustrative purposes only and are not to be construed as limiting the disclosure.

### Example 1: Reducing Immunogenicity to Recombinant Adeno-Associated Virus (rAAV)

In certain experiments a CD32B×CD79B bi-specific antibody can be administered—as monotherapy or in combination with other immune-modulators, for example sirolimus, rapamycin, abatacept, teplizumab and immunoglobulin G-degrading enzyme of Streptococcus pyogenes- to mice prior to administration of a rAAV vector encoding a potentially therapeutic transgene, and at subsequent points thereafter to maintain pharmacological coverage. At specific time points (e.g., 15-45 days) mice can be euthanized, and immunological assessments and efficiency of adeno-associated virus gene transfer can be evaluated. Immunological endpoints include: Total antibody (IgM, IgG) against the rAAV vector and transgene, respectively, complement activation, B cell and T cell functional assays against vector and transgenes, and phenotypic characterization. Efficiency of the adeno-associated virus gene transfer measures include blood vector genome copy number by PCR, and transgene activity in tissues including, but not limited to, heart, skeletal muscle, liver and spleen.

Results achieved with administration of CD32B×CD79B bi-specific antibody to rAAV recipient animals, compared to placebo control, can include diminishment of anti-rAAV and transgene specific antibody responses, decreased complement activation and reduction in anti-rAAV specific T cell activity. Vector genome copy number and transgene activity can be increased with administration of CD32B×CD79B bi-specific antibody compared to placebo animals, supporting the hypothesis that administration of CD32B×CD79B bi-specific antibody reduces the immunogenicity of recombinant AAV.

### Example 2: Reducing Immunogenicity to Repeat Dosages of Recombinant Adeno-Associated Virus (rAAV)

In certain experiments a CD32B×CD79B bi-specific antibody can be administered—as monotherapy or in combination with other immune-modulators, for example sirolimus- to mice prior to administration of an rAAV vector encoding a potentially therapeutic transgene and at subsequent points thereafter to maintain pharmacological coverage. At specific time points (e.g., 45, 90, 135 days), mice can receive an additional administration(s) of the same rAAV vector/transgene. Mice can continue to receive pharmacologically relevant doses of CD32B×CD79B bi-specific antibody prior to being euthanized at certain time point (e.g., 90, 135, 180 days) and assessment of immunological endpoints and efficiency of adeno-associated virus gene transfer. Immunological endpoints measured include: Total antibody against the rAAV vector and transgene, respectively; complement activation, B cell and T cell functional assays against vector and transgenes, and phenotypic characterization. Efficiency of the adeno-associated virus gene transfer measures include vector genome copy number by PCR and transgene activity in various tissues including, but not limited to, heart, skeletal muscle, liver and spleen.

Results achieved with administration of CD32B×CD79B bi-specific antibody to rAAV recipient animals, compared to placebo control, can include diminishment of anti-rAAV and transgene specific antibody responses, decreased complement activation and reduction in anti-rAAV specific T cell activity. Vector genome copy number and transgene activity can be increased with administration of CD32B×CD79B bi-specific antibody compared to placebo animals. These effects can be noted after single administration of rAAV vector and after subsequent administration(s) of rAAV vector, supporting the hypothesis that administration of CD32B×CD79B bi-specific antibody may allow for repeat dosing and increased efficacy of immunogenic recombinant AAV.

### Example 3: Reducing Pre-Existing Immune Response to AAV or rAAV Prior to Administration of Recombinant Adeno-Associated Virus

In certain experiments pre-existing immunity to wild-type AAV or rAAV can be developed in mice by administration of the respective AAV or rAAV of the same AAV serotype, encoding a potentially therapeutic transgene. Subsequently, at a specific time point, e.g., Day 15, CD32B×CD79B bi-specific antibody can be administered—as monotherapy or in combination with other immune-modulators, for example sirolimus- to the same mice for a specific period of time, e.g., 14 days prior to re administration of the same rAAV vector encoding the potentially therapeutic transgene and at subsequent points thereafter to maintain pharmacological coverage. At specific time points (e.g., 45, 90, 135 days), some mice can receive an additional administration(s) of the same rAAV vector/transgene. Those mice can continue to receive pharmacologically relevant doses of CD32B×CD79B bi-specific antibody prior to being euthanized at certain time point (e.g., 90, 135, 180 days) and assessment of immunological endpoints and efficiency of adeno-associated virus gene transfer. Immunological endpoints measured include: Total antibody against the wild-type AAV and/or rAAV vector and transgene, respectively; complement activation, B cell and T cell functional assays against AAV and/or vector and transgenes, and phenotypic characterization. Efficiency of the adeno-associated virus gene transfer measures include vector genome copy number by PCR and transgene activity in tissues including, but not limited to, heart, skeletal muscle, liver and spleen.

Results achieved with administration of CD32B×CD79B bi-specific antibody to AAV and/or rAAV pre-immune animals, compared to placebo control, can include diminishment of pre-existing anti-AAV and/or rAAV and transgene specific antibody responses, decreased complement activation and reduction in anti-rAAV specific T cell activity. After subsequent administration of rAAV, diminishment of anti-rAAV and transgene specific antibody responses, decreased complement activation and reduction in anti-rAAV specific T cell activity can be noted. Vector genome copy number and transgene activity can be increased with administration of CD32B×CD79B bi-specific antibody compared to placebo animals. These effects can be noted after single administration of rAAV vector to previously immune animals and after subsequent administration(s) of rAAV vector to previously immune animals, supporting the hypothesis that administration of CD32B×CD79B bi-specific antibody may allow for dosing of immunogenic recombinant AAV where pre-existing immune response to AAV or rAAV is present.

### Example 4: Reducing Immunogenicity to Repeat Doses of Enzyme Replacement Therapy (ERT)

In certain experiments a CD32B×CD79B bi-specific antibody can be administered—as monotherapy or in combination with other immune-modulators, for example sirolimus- to mice with an inherent defect in a certain enzyme (such as knockout mice disclosed in Front Immunol. 2019 Mar. 13; 10:416, incorporated herein by reference) prior to administration of the enzyme replacement therapy and at subsequent points thereafter to maintain pharmacological coverage. At specific time points (e.g., 7, 14, 21, 28 days etc.), mice can receive an additional administration(s) of the same ERT. Mice can continue to receive pharmacologically relevant doses of CD32B×CD79B bi-specific antibody prior to being euthanized at certain time point (e.g., 14, 21, 28, 35 days etc.) and assessment of immunological endpoints and efficiency of enzyme replacement therapy. Immunological endpoints include: 1) Total antibody (IgM, IgG) against the enzyme, B cell functional assays and phenotypic characterization; and 2) Efficiency of the enzyme transfer measures include reversal of the physiological consequences of the enzyme defect and biochemical analysis of enzyme and substrate activity throughout the experiment.

Results achieved with administration of CD32B×CD79B bi-specific antibody to enzyme replacement recipient animals, compared to placebo control, can include diminishment of anti-enzyme specific antibody responses, improvement in enzyme dependent physiological outcomes, increased duration of enzyme activity and a reduction in substrate accumulation observed with administration of CD32B×CD79B bi-specific antibody compared to placebo animals, supporting the hypothesis that administration of CD32B×CD79B bi-specific antibody may decrease immunogenicity to enzyme replacement therapy and allow for repeat dosing and increased efficacy of enzyme replacement therapy.

### Example 5: Reducing Immunogenicity to Repeat Dosing of Antibody- and Fusion Protein-Based Therapeutics

In certain experiments a CD32B×CD79B bi-specific antibody can be administered—as monotherapy or in combination with other immune-modulators, for example sirolimus- to mice prior to administration of an antibody or fusion protein, analogous to a human antibody- or fusion protein-based therapy, and at subsequent points thereafter to maintain pharmacological coverage At specific time points (e.g., 7, 14, 21, 28 days etc.), mice can receive an additional administration(s) of the same antibody or fusion protein. Mice can continue to receive pharmacologically relevant doses of CD32B×CD79B bi-specific antibody prior to being euthanized at certain time point (e.g., 14, 21, 28, 35 days, etc.) and assessment of immunological endpoints and activity of the antibody or fusion protein. Immunological endpoints include: 1) Total antibody (IgM, IgG) against the enzyme, B cell functional assays and phenotypic characterization; and 2) Efficiency of the antibody or fusion protein measures include pharmacokinetic, immunological and/or pharmacodynamic analysis of the activity of the antibody or fusion protein throughout the experiment, e.g., ability of the antibody or fusion protein to inhibit its target protein.

Results achieved with administration of CD32B×CD79B bi-specific antibody to antibody or fusion protein recipient animals, compared to placebo control, can include diminishment of anti-antibody or fusion protein antibody responses, decreased clearance and an increased half-life (t½). Improved and prolonged pharmacodynamic measures of efficacy can also be observed compared to placebo animals, supporting the hypothesis that administration of CD32B×CD79B bi-specific antibody may allow for repeat dosing and increased efficacy of immunogenic antibody or fusion proteins.

### Example 6: A Phase 1b, Double-Blind, Placebo-Controlled, Multiple Ascending Dose Study to Evaluate the Safety, Tolerability, Pharmacokinetics, Pharmacodynamics, and Immunogenicity of PRV-3279 in Healthy Subjects

In this study, the safety, tolerability, and immunogenicity of multiple doses of PRV-3279 were assessed in healthy subjects at dose levels projected to provide sustained high level of receptor coverage. Healthy subjects were selected for this study to avoid background medications that could confound the development of ADA and signs and symptoms that could confound assessment of tolerability, thus allowing for a more thorough and safe examination of the immunogenicity and tolerability of repeat doses of PRV-3279.

Two cohorts were planned for sequential enrollment. Cohort A evaluated PRV-3279 3 mg/kg every 2 weeks for a total of 3 doses. Cohort B evaluated PRV-3279 10 mg/kg every 2 weeks for 3 doses. Each cohort was composed of 8 subjects randomly assigned to either PRV-3279 or placebo at a ratio of 3:1 (i.e., n=6 for PRV-3279 and n=2 for placebo). The 3 doses of study drug (PRV-3279 or placebo) were administered as a 2-hour IV infusion on Day 1, Day 15, and Day 29 in each cohort.

Subjects underwent screening evaluations to determine eligibility within 28 days before randomization and the first dose administration on Day 1. On Day −1, subjects were admitted to the clinical research unit (CRU) and underwent baseline tests to confirm their eligibility. On Day 1, each subject was randomly assigned to receive a 2-hour IV infusion of either PRV-3279 or placebo in a double-blind fashion and monitored for 4 hours after dosing. On Day 2, the subjects had safety laboratory tests, PK, and evaluation for AEs and were discharged from the CRU. The subjects returned to the CRU to receive the second dose (Day 15) and third dose (Day 29) of their assigned treatment. Similar to the first dose, subjects were admitted into the CRU on the day before dosing and discharged on the day after dosing.

Each cohort included 2 sentinel subjects: 1 received PRV-3279 and 1 received placebo in a double-blind fashion. Sentinel subjects were assessed for AEs (e.g., infusion reactions, delayed hypersensitivity) from the start of the first infusion through at least Day 7 before the remaining subjects in the cohort received their first infusion. The use of sentinel subjects and the staggered dosing schedule ensured that any potential and high-frequency reaction (e.g., infusion reactions related to ADA) would be recognized before repeated dosing of the full cohort.

Safety assessments included reported AEs, including hypersensitivity or infusion reaction, vital sign measurements, physical examinations, ECGs, and clinical laboratory tests. Physical examinations were performed to establish a baseline and confirm physical signs associated with AEs. Adverse events were collected at each visit and evaluated for severity and relatedness to the study drug. On Day 1, Day 15, and Day 29, vital signs (temperature, pulse, blood pressure, and respiratory rate) were recorded immediately at time 0 (pre-dose; up to 5 minutes before the infusion), 0.5 hour, 1 hour (midpoint of infusion), 2 hours (end of infusion), and 6 hours after the start of infusion (4 hours after the end of infusion). The start of IV infusion was designated as time “0” hour. Vital signs were obtained at ±5 minutes of the planned time points. Height was recorded at the Screening Visit only. Weight was obtained on Day −1, Day 14, and Day 28.

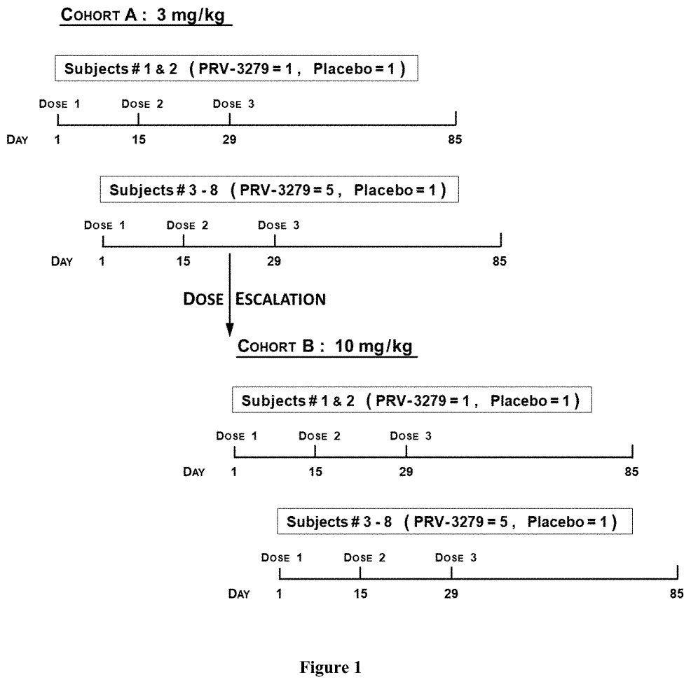

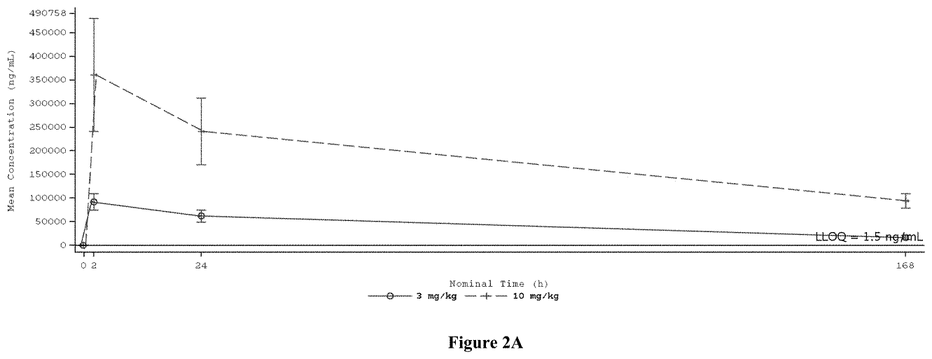

Serum samples for PK, immunogenicity, and PD were obtained at the selected time points. A diagram of the study design is provided in FIG. 1.

### Summary of Adverse Events:

There were no AESIs, serious TEAEs, SAEs, or TEAEs leading to death during the study. Four mild but recurrent TEAEs led to the withdrawal of 1 (16.7%) PRV-3279 10 mg/kg subject. No other TEAEs led to subject withdrawal from the study (Table Error! No text of specified style in document.).

Overall, 34 TEAEs were reported by 9 (56.3%) subjects. Eighteen TEAEs were reported in 5 (83.3%) PRV-3279 10 mg/kg subjects; 12 TEAEs were reported in 3 (50.0%) PRV-3279 3 mg/kg subjects; and 4 TEAEs were reported in 1 (25.0%) placebo subject (Table Error! No text of specified style in document.). Twelve TEAEs in 4 (66.7%) PRV-3279 10 mg/kg subjects and 4 TEAEs in 1 (16.7%) PRV-3279 3 mg/kg subject were considered by the Investigator to be related to the study drug; all other reported TEAEs were considered unrelated (Table Error! No text of specified style in document.).

Table Error! No text of specified style in document.
Summary of Treatment-Emergent Adverse
Events by Treatment and Overall (Safety Population)

| | | | |
|---|---|---|---|
| PRV-3279 | PRV-3279 | Pooled | |
| 3 mg/kg | 10 mg/kg | Placebo | Total |
| (N = 6) | (N = 6) | (N = 4) | (N = 16) |
| n (%) E | n (%) E | n (%) E | n (%) E |

Number (%) of subjects with:

| | | | | |
|---|---|---|---|---|
| At least 1 TEAE | 3 (50.0) 12 | 5 (83.3) 18 | 1 (25.0) 4 | 9 (56.3) 34 |
| At least 1 related TEAE | 1 (16.7) 4 | 4 (66.7) 12 | 0 | 5 (31.3) 16 |
| At least 1 unrelated TEAE | 3 (50.0) 8 | 4 (66.7) 6 | 1 (25.0) 4 | 8 (50.0) 18 |
| At least 1 serious TEAE | 0 | 0 | 0 | 0 |
| At least 1 serious related | 0 | 0 | 0 | 0 |
| TEAE | | | | |
| TEAEs leading to | 0 | 1 (16.7) 4 | 0 | 1 (6.3) 4 |
| withdrawal from study | | | | |
| TEAEs leading to | 0 | 0 | 0 | 0 |
| withdrawal from study | | | | |
| drug but remaining in | | | | |
| the study | | | | |
| TEAEs leading to death | 0 | 0 | 0 | 0 |
| TEAEs with at least 1 | 0 | 0 | 0 | 0 |
| AESI | | | | |

AE = adverse event;
AESI = adverse event of special interest;
E = number of events;
MedDRA = Medical Dictionary for Regulatory Activities;
N = total number of subjects;
n = number of subjects;
% = percentage of subjects (the denominator was N);
TEAE = treatment-emergent adverse event
All AEs were coded using MedDRA dictionary version 22.0.

Two AEs considered by the Investigator to be non-TEAEs were reported in 2 (9.2%) subjects during the study; both were considered unrelated to the study drug (Table 2).

TABLE 2
Summary of Non-Treatment-Emergent
Adverse Events (All Screened Subjects)

| | Total |
|---|---|
| | (N = 70) |
| | n (%) E |
| Number (%) of subjects with: | |
| At least 1 non-TEAE | 2 (2.9) 2 |
| At least 1 related non-TEAE | 0 |
| At least 1 unrelated non-TEAE | 2 (2.9) 2 |
| At least 1 serious non-TEAE | 0 |
| At least 1 serious related non-TEAE | 0 |
| Non-TEAEs leading to withdrawal from | 0 |
| study | |
| Non-TEAEs leading to withdrawal from | 0 |
| study drug but remaining in the study | |
| Non-TEAEs leading to death | 0 |

E = number of events;
MedDRA = Medical Dictionary for Regulatory Activities;
N = total number of subjects;
n = number of subjects;
% = percentage of subjects (the denominator was N);
TEAE = treatment-emergent adverse event
All AEs were coded using MedDRA dictionary version 22.0.

A summary of TEAEs by treatment and overall, by SOC and PT is presented in Table. A summary of TEAEs by SOC and PT by treatment by severity is presented in Table and a summary of related TEAEs is presented in Table. A summary of TEAEs leading to discontinuation by SOC and PT, by treatment and overall is presented in Table.

TABLE 3
Summary of Treatment-Emergent Adverse Events by Treatment and
Overall, by System Organ Class and Preferred Term (Safety Population)

| | PRV-3279 | PRV-3279 | Pooled | |
|---|---|---|---|---|
| | 3 mg/kg | 10 mg/kg | Placebo | Total |
| System Organ Class/ | (N = 6) | (N = 6) | (N = 4) | (N = 16) |
| Preferred Term | n (%) E | n (%) E | n (%) E | n (%) E |
| Number of subjects | 3 (50.0) 12 | 5 (83.3) 18 | 1 (25.0) 4 | 9 (56.3) 34 |
| and events with at | | | | |
| least 1 TEAE | | | | |
| General disorders and | 3 (50.0) 6 | 2 (33.3) 4 | 1 (25.0) 3 | 6 (37.5) 13 |
| administration site | | | | |
| conditions | | | | |
| Catheter site pain | 1 (16.7) 1 | 0 | 1 (25.0) 1 | 2 (12.5) 2 |
| Feeling hot | 0 | 2 (33.3) 3 | 0 | 2 (12.5) 3 |
| Vessel puncture site | 1 (16.7) 3 | 1 (16.7) 1 | 0 | 2 (12.5) 4 |
| bruise | | | | |
| Catheter site erythema | 0 | 0 | 1 (25.0) 1 | 1 (6.3) 1 |
| Chills | 1 (16.7) 1 | 0 | 0 | 1 (6.3) 1 |
| Fatigue | 1 (16.7) 1 | 0 | 0 | 1 (6.3) 1 |
| Non-cardiac chest pain | 0 | 0 | 1 (25.0) 1 | 1 (6.3) 1 |
| Skin and subcutaneous | 1 (16.7) 2 | 3 (50.0) 6 | 0 | 4 (25.0) 8 |
| tissue disorders | | | | |
| Dermatitis contact | 1 (16.7) 1 | 1 (16.7) 1 | 0 | 2 (12.5) 2 |
| Cold sweat | 0 | 1 (16.7) 2 | 0 | 1 (6.3) 2 |
| Hyperhidrosis | 0 | 1 (16.7) 1 | 0 | 1 (6.3) 1 |
| Night sweats | 1 (16.7) 1 | 0 | 0 | 1 (6.3) 1 |
| Pruritus | 0 | 1 (16.7) 1 | 0 | 1 (6.3) 1 |
| Xeroderma | 0 | 1 (16.7) 1 | 0 | 1 (6.3) 1 |
| Nervous system | 1 (16.7) 1 | 1 (16.7) 1 | 1 (25.0) 1 | 3 (18.8) 3 |
| disorders | | | | |
| Dizziness | 0 | 0 | 1 (25.0) 1 | 1 (6.3) 1 |
| Headache | 0 | 1 (16.7) 1 | 0 | 1 (6.3) 1 |
| Somnolence | 1 (16.7) 1 | 0 | 0 | 1 (6.3) 1 |
| Gastrointestinal | 1 (16.7) 1 | 1 (16.7) 2 | 0 | 2 (12.5) 3 |
| disorders | | | | |
| Abdominal discomfort | 0 | 1 (16.7) 1 | 0 | 1 (6.3) 1 |
| Abdominal pain | 0 | 1 (16.7) 1 | 0 | 1 (6.3) 1 |
| Nausea | 1 (16.7) 1 | 0 | 0 | 1 (6.3) 1 |
| Respiratory, thoracic | 0 | 2 (33.3) 2 | 0 | 2 (12.5) 2 |
| and mediastinal | | | | |
| disorders | | | | |
| Dyspnoea | 0 | 1 (16.7) 1 | 0 | 1 (6.3) 1 |
| Oropharyngeal pain | 0 | 1 (16.7) 1 | 0 | 1 (6.3) 1 |
| Ear and labyrinth | 0 | 1 (16.7) 1 | 0 | 1 (6.3) 1 |
| disorders | | | | |
| Ear pain | 0 | 1 (16.7) 1 | 0 | 1 (6.3) 1 |
| Infections and | 0 | 1 (16.7) 1 | 0 | 1 (6.3) 1 |
| infestations | | | | |
| Respiratory tract | 0 | 1 (16.7) 1 | 0 | 1 (6.3) 1 |
| infection viral | | | | |
| Injury, poisoning | 1 (16.7) 1 | 0 | 0 | 1 (6.3) 1 |
| and procedural | | | | |
| complications | | | | |
| Limb injury | 1 (16.7) 1 | 0 | 0 | 1 (6.3) 1 |
| Musculoskeletal and | 1 (16.7) 1 | 0 | 0 | 1 (6.3) 1 |
| connective tissue | | | | |
| disorders | | | | |
| Back pain | 1 (16.7) 1 | 0 | 0 | 1 (6.3) 1 |
| Reproductive system | 0 | 1 (16.7) 1 | 0 | 1 (6.3) 1 |
| and breast disorders | | | | |
| Metrorrhagia | 0 | 1 (16.7) 1 | 0 | 1 (6.3) 1 |

E = number of events;
MedDRA = Medical Dictionary for Regulatory Activities;
N = total number of subjects;
n = number of subjects;
% = percentage of subjects (the denominator was N);
TEAE = treatment-emergent adverse event
Subjects with more than 1 TEAE in a given system organ class and preferred term were counted only once in that category.
System organ classes and preferred terms were sorted decreasingly by subject counts in total column and then alphabetically.
All AEs were coded using MedDRA dictionary version 22.0.

TABLE 4
Summary of Treatment-Emergent Adverse Events by System Organ Class and
Preferred Term by Treatment and by Severity (Safety Population)

| | PRV-3279 | PRV-3279 | Pooled | |
|---|---|---|---|---|
| | 3 mg/kg | 10 mg/kg | Placebo | Total |
| System Organ Class/ | (N = 6) | (N = 6) | (N = 4) | (N = 16) |
| Preferred Term/Severity | n (%) E | n (%) E | n (%) E | n (%) E |
| Number of subjects and events with at least | 3 (50.0) 12 | 5 (83.3) 18 | 1 (25.0) 4 | 9 (56.3) 34 |
| 1 TEAE | | | | |

TEAEs severity

| | | | | |
|---|---|---|---|---|
| Mild | 2 (33.3) 11 | 4 (66.7) 17 | 1 (25.0) 4 | 7 (43.8) 32 |
| Moderate | 1 (16.7) 1 | 1 (16.7) 1 | 0 | 2 (12.5) 2 |
| Severe | 0 | 0 | 0 | 0 |
| General disorders and administration site | 3 (50.0) 6 | 2 (33.3) 4 | 1 (25.0) 3 | 6 (37.5) 13 |
| conditions | | | | |
| Catheter site pain | 1 (16.7) 1 | 0 | 1 (25.0) 1 | 2 (12.5) 2 |
| Mild | 1 (16.7) 1 | 0 | 1 (25.0) 1 | 2 (12.5) 2 |
| Feeling hot | 0 | 2 (33.3) 3 | 0 | 2 (12.5) 3 |
| Mild | 0 | 2 (33.3) 3 | 0 | 2 (12.5) 3 |
| Vessel puncture site bruise | 1 (16.7) 3 | 1 (16.7) 1 | 0 | 2 (12.5) 4 |
| Mild | 1 (16.7) 3 | 1 (16.7) 1 | 0 | 2 (12.5) 4 |
| Catheter site erythema | 0 | 0 | 1 (25.0) 1 | 1 (6.3) 1 |
| Mild | 0 | 0 | 1 (25.0) 1 | 1 (6.3) 1 |
| Chills | 1 (16.7) 1 | 0 | 0 | 1 (6.3) 1 |
| Mild | 1 (16.7) 1 | 0 | 0 | 1 (6.3) 1 |
| Fatigue | 1 (16.7) 1 | 0 | 0 | 1 (6.3) 1 |
| Mild | 1 (16.7) 1 | 0 | 0 | 1 (6.3) 1 |
| Non-cardiac chest pain | 0 | 0 | 1 (25.0) 1 | 1 (6.3) 1 |
| Mild | 0 | 0 | 1 (25.0) 1 | 1 (6.3) 1 |
| Skin and subcutaneous tissue disorders | 1 (16.7) 2 | 3 (50.0) 6 | 0 | 4 (25.0) 8 |
| Dermatitis contact | 1 (16.7) 1 | 1 (16.7) 1 | 0 | 2 (12.5) 2 |
| Mild | 0 | 1 (16.7) 1 | 0 | 1 (6.3) 1 |
| Moderate | 1 (16.7) 1 | 0 | 0 | 1 (6.3) 1 |
| Cold sweat | 0 | 1 (16.7) 2 | 0 | 1 (6.3) 2 |
| Mild | 0 | 1 (16.7) 2 | 0 | 1 (6.3) 2 |
| Hyperhidrosis | 0 | 1 (16.7) 1 | 0 | 1 (6.3) 1 |
| Mild | 0 | 1 (16.7) 1 | 0 | 1 (6.3) 1 |
| Night sweats | 1 (16.7) 1 | 0 | 0 | 1 (6.3) 1 |
| Mild | 1 (16.7) 1 | 0 | 0 | 1 (6.3) 1 |
| Pruritus | 0 | 1 (16.7) 1 | 0 | 1 (6.3) 1 |
| Mild | 0 | 1 (16.7) 1 | 0 | 1 (6.3) 1 |
| Xeroderma | 0 | 1 (16.7) 1 | 0 | 1 (6.3) 1 |
| Mild | 0 | 1 (16.7) 1 | 0 | 1 (6.3) 1 |
| Nervous system disorders | 1 (16.7) 1 | 1 (16.7) 1 | 1 (25.0) 1 | 3 (18.8) 3 |
| Dizziness | 0 | 0 | 1 (25.0) 1 | 1 (6.3) 1 |
| Mild | 0 | 0 | 1 (25.0) 1 | 1 (6.3) 1 |
| Headache | 0 | 1 (16.7) 1 | 0 | 1 (6.3) 1 |
| Moderate | 0 | 1 (16.7) 1 | 0 | 1 (6.3) 1 |
| Somnolence | 1 (16.7) 1 | 0 | 0 | 1 (6.3) 1 |
| Mild | 1 (16.7) 1 | 0 | 0 | 1 (6.3) 1 |
| Gastrointestinal disorders | 1 (16.7) 1 | 1 (16.7) 2 | 0 | 2 (12.5) 3 |
| Abdominal discomfort | 0 | 1 (16.7) 1 | 0 | 1 (6.3) 1 |
| Mild | 0 | 1 (16.7) 1 | 0 | 1 (6.3) 1 |
| Abdominal pain | 0 | 1 (16.7) 1 | 0 | 1 (6.3) 1 |
| Mild | 0 | 1 (16.7) 1 | 0 | 1 (6.3) 1 |
| Nausea | 1 (16.7) 1 | 0 | 0 | 1 (6.3) 1 |
| Mild | 1 (16.7) 1 | 0 | 0 | 1 (6.3) 1 |
| Respiratory, thoracic and mediastinal | 0 | 2 (33.3) 2 | 0 | 2 (12.5) 2 |
| disorders | | | | |
| Dyspnoea | 0 | 1 (16.7) 1 | 0 | 1 (6.3) 1 |
| Mild | 0 | 1 (16.7) 1 | 0 | 1 (6.3) 1 |
| Oropharyngeal pain | 0 | 1 (16.7) 1 | 0 | 1 (6.3) 1 |
| Mild | 0 | 1 (16.7) 1 | 0 | 1 (6.3) 1 |
| Ear and labyrinth disorders | 0 | 1 (16.7) 1 | 0 | 1 (6.3) 1 |
| Ear pain | 0 | 1 (16.7) 1 | 0 | 1 (6.3) 1 |
| Mild | 0 | 1 (16.7) 1 | 0 | 1 (6.3) 1 |
| Infections and infestations | 0 | 1 (16.7) 1 | 0 | 1 (6.3) 1 |
| Respiratory tract infection viral | 0 | 1 (16.7) 1 | 0 | 1 (6.3) 1 |
| Mild | 0 | 1 (16.7) 1 | 0 | 1 (6.3) 1 |
| Injury, poisoning and procedural | 1 (16.7) 1 | 0 | 0 | 1 (6.3) 1 |
| complications | | | | |
| Limb injury | 1 (16.7) 1 | 0 | 0 | 1 (6.3) 1 |
| Mild | 1 (16.7) 1 | 0 | 0 | 1 (6.3) 1 |
| Musculoskeletal and connective tissue | 1 (16.7) 1 | 0 | 0 | 1 (6.3) 1 |
| disorders | | | | |
| Back pain | 1 (16.7) 1 | 0 | 0 | 1 (6.3) 1 |
| Mild | 1 (16.7) 1 | 0 | 0 | 1 (6.3) 1 |
| Reproductive system and breast disorders | 0 | 1 (16.7) 1 | 0 | 1 (6.3) 1 |
| Metrorrhagia | 0 | 1 (16.7) 1 | 0 | 1 (6.3) 1 |
| Mild | 0 | 1 (16.7) 1 | 0 | 1 (6.3) 1 |

E = number of events;
MedDRA = Medical Dictionary for Regulatory Activities;
N = total number of subjects;
n = number of subjects;
% = percentage of subjects (the denominator was N);
TEAE = treatment-emergent adverse event
A subject was presented once for each system organ class and preferred term according to their worst severity.
System organ classes and preferred terms were sorted decreasingly by subject counts in total column and then alphabetically.
All AEs were coded using MedDRA dictionary version 22.0.

TABLE 5
Summary of Related Treatment-Emergent Adverse Events by System Organ
Class and Preferred Term by Treatment (Safety Population)

| | PRV-3279 | PRV-3279 | Pooled | |
|---|---|---|---|---|
| | 3 mg/kg | 10 mg/kg | Placebo | Total |
| System Organ Class/ | (N = 6) | (N = 6) | (N = 4) | (N = 16) |
| Preferred Term | n (%) E | n (%) E | n (%) E | n (%) E |
| Number of subjects and events with at least | 3 (50.0) 12 | 5 (83.3) 18 | 1 (25.0) 4 | 9 (56.3) 34 |
| 1 TEAE | | | | |
| Number of subjects and TEAEs considered | 1 (16.7) 4 | 4 (66.7) 12 | 0 | 5 (31.3) 16 |
| related | | | | |
| General disorders and administration site | 3 (50.0) 6 | 2 (33.3) 4 | 1 (25.0) 3 | 6 (37.5) 13 |
| conditions | | | | |
| Feeling hot | 0 | 2 (33.3) 3 | 0 | 2 (12.5) 3 |
| Chills | 1 (16.7) 1 | 0 | 0 | 1 (6.3) 1 |
| Fatigue | 1 (16.7) 1 | 0 | 0 | 1 (6.3) 1 |
| Skin and subcutaneous tissue disorders | 1 (16.7) 2 | 3 (50.0) 6 | 0 | 4 (25.0) 8 |
| Cold sweat | 0 | 1 (16.7) 2 | 0 | 1 (6.3) 2 |
| Hyperhidrosis | 0 | 1 (16.7) 1 | 0 | 1 (6.3) 1 |
| Night sweats | 1 (16.7) 1 | 0 | 0 | 1 (6.3) 1 |
| Nervous system disorders | 1 (16.7) 1 | 1 (16.7) 1 | 1 (25.0) 1 | 3 (18.8) 3 |
| Headache | 0 | 1 (16.7) 1 | 0 | 1 (6.3) 1 |
| Somnolence | 1 (16.7) 1 | 0 | 0 | 1 (6.3) 1 |
| Gastrointestinal disorders | 1 (16.7) 1 | 1 (16.7) 2 | 0 | 2 (12.5) 3 |
| Abdominal discomfort | 0 | 1 (16.7) 1 | 0 | 1 (6.3) 1 |
| Abdominal pain | 0 | 1 (16.7) 1 | 0 | 1 (6.3) 1 |
| Respiratory, thoracic and mediastinal | 0 | 2 (33.3) 2 | 0 | 2 (12.5) 2 |
| disorders | | | | |
| Dyspnoea | 0 | 1 (16.7) 1 | 0 | 1 (6.3) 1 |
| Oropharyngeal pain | 0 | 1 (16.7) 1 | 0 | 1 (6.3) 1 |
| Reproductive system and breast disorders | 0 | 1 (16.7) 1 | 0 | 1 (6.3) 1 |
| Metrorrhagia | 0 | 1 (16.7) 1 | 0 | 1 (6.3) 1 |

E = number of events;
MedDRA = Medical Dictionary for Regulatory Activities;
N = total number of subjects;
n = number of subjects;
% = percentage of subjects (the denominator was N);
TEAE = treatment-emergent adverse event
A subject was presented once for each system organ class and preferred term according to their worst causality.
System organ classes and preferred terms were sorted decreasingly by subject counts in total column and then alphabetically.
All AEs were coded using MedDRA dictionary version 22.0.

TABLE 6
Summary of Treatment-Emergent Adverse Events Leading to
Discontinuation by System Organ Class and Preferred Term,
by Treatment and Overall (Safety Population)

| | PRV-3279 | PRV-3279 | Pooled | |
|---|---|---|---|---|
| | 3 mg/kg | 10 mg/kg | Placebo | Total |
| System Organ Class/ | (N = 6) | (N = 6) | (N = 4) | (N = 16) |
| Preferred Term | n (%) E | n (%) E | n (%) E | n (%) E |
| Number of subjects and | 0 | 1 (16.7) 4 | 0 | 1 (6.3) 4 |
| events of TEAEs leading | | | | |
| to discontinuation | | | | |
| Gastrointestinal disorders | 0 | 1 (16.7) 1 | 0 | 1 (6.3) 1 |
| Abdominal pain | 0 | 1 (16.7) 1 | 0 | 1 (6.3) 1 |
| General disorders and | 0 | 1 (16.7) 1 | 0 | 1 (6.3) 1 |
| administration site | | | | |
| conditions | | | | |
| Feeling hot | 0 | 1 (16.7) 1 | 0 | 1 (6.3) 1 |
| Skin and subcutaneous | 0 | 1 (16.7) 2 | 0 | 1 (6.3) 2 |
| tissue disorders | | | | |
| Cold sweat | 0 | 1 (16.7) 1 | 0 | 1 (6.3) 1 |
| Hyperhidrosis | 0 | 1 (16.7) 1 | 0 | 1 (6.3) 1 |

E = number of events. MedDRA = Medical Dictionary for Regulatory Activities; N = total number of subjects; n = number of subjects; % = percentage of subjects (the denominator was N); TEAE = treatment-emergent adverse event
A subject was presented once for each system organ class and preferred term according to their worst causality.
System organ classes and preferred terms were sorted decreasingly by subject counts in total column and then alphabetically.
All AEs were coded using MedDRA dictionary version 22.0.

### Pharmacokinetic Concentration Data

PRV-3279 is quantitatively measured from human serum using ECL. In this assay, an uncoated MSD Multi-Array® Standard-Bind plate is coated with rabbit anti-h8B5 antibody as a capture reagent for PRV-3279. Samples containing PRV-3279 are incubated on the coated plate. The bound PRV-3279 is detected with biotinylated 2A5 antibody. The Streptavidin Sulfo-Tag conjugate is added and binds to the primary detection antibody. Tripropylamine (TPA, MSD Gold Read Buffer) is added to the plate, and upon application of an electrical charge, an electrochemiluminescent signal is produced and detected with a MSD SECTOR S 600 plate reader.

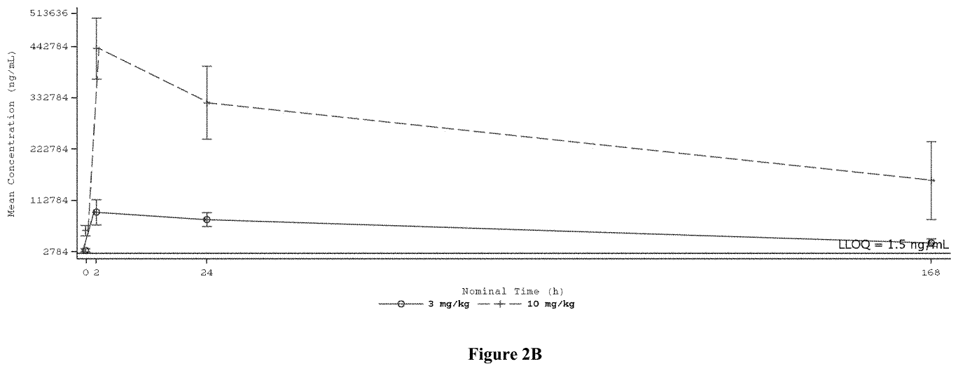

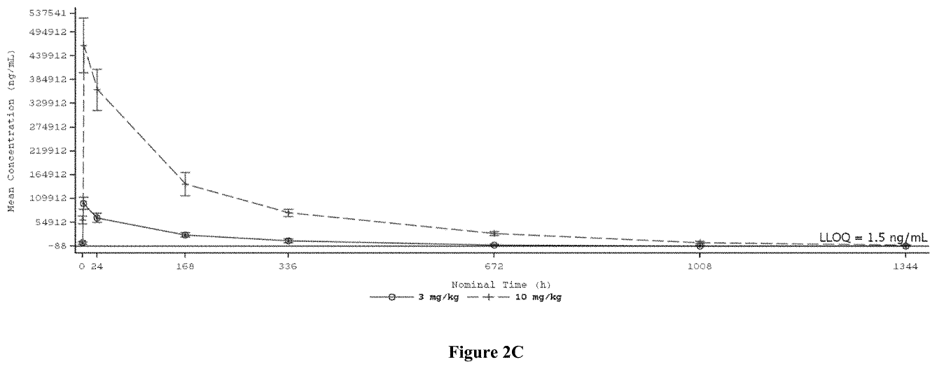

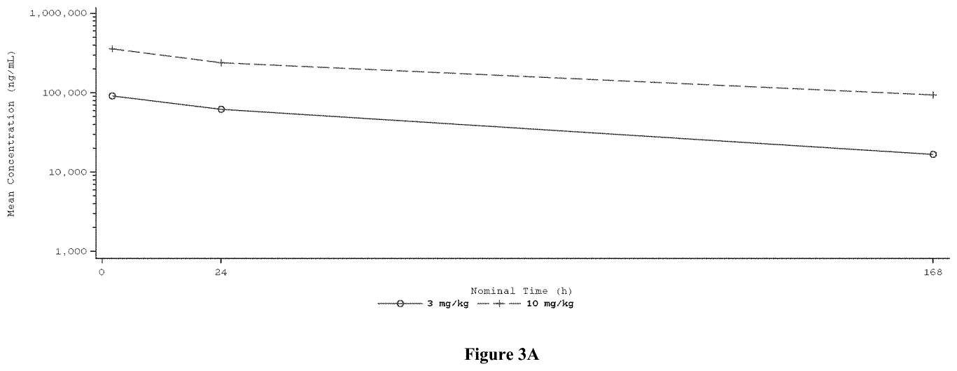

The arithmetic mean (±SD) PRV-3279 serum concentration-time data are displayed in FIGS. 2A-2C. BLQ=below the limit of quantification; LLOQ; lower limit of quantification; SD=standard deviation. Error bars: SD. Values that were BLQ at pre-dose and in the absorption phase before the first quantifiable concentration were substituted by zeros. Thereafter, BLQ values between evaluable concentrations were substituted by LLOQ/2. LLOQ=1.5 ng/mL.

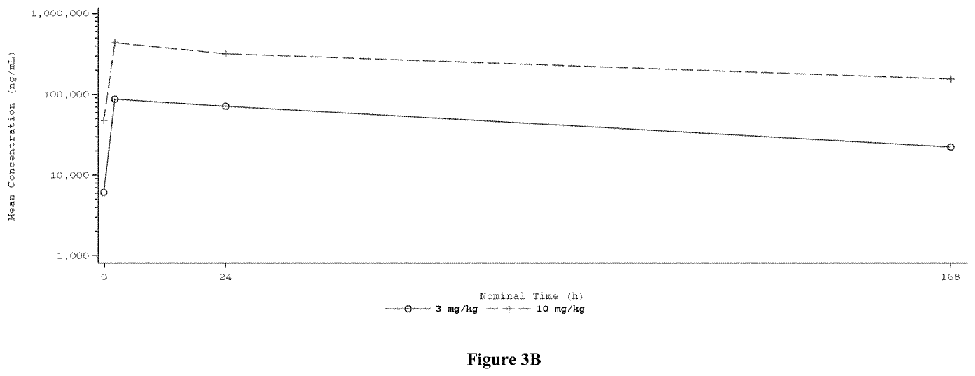

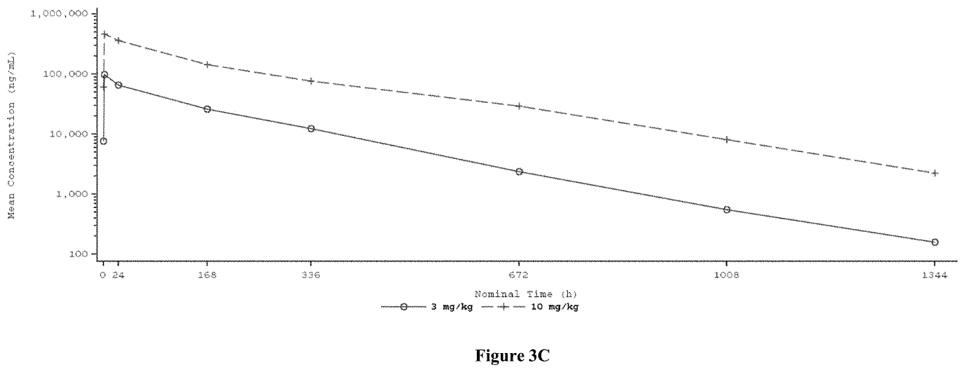

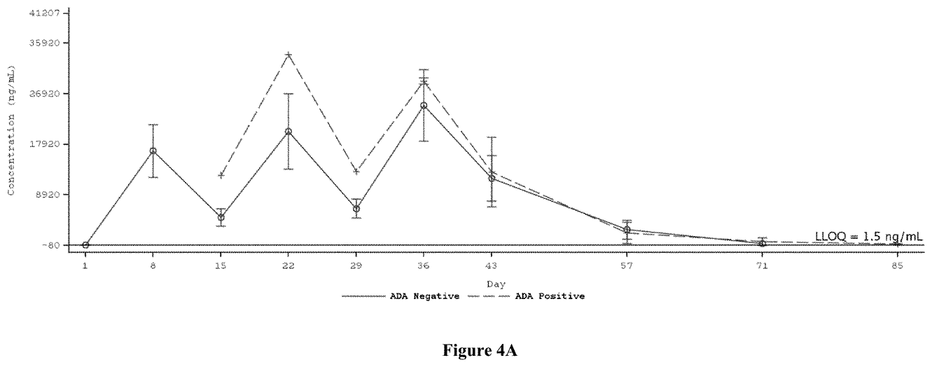

The arithmetic mean PRV-3279 serum concentration-time data are displayed in FIGS. 3A-3C. BLQ=below the limit of quantification; LLOQ; lower limit of quantification; SD=standard deviation. Values that were BLQ at pre-dose and in the absorption phase before the first quantifiable concentration were substituted by zeros. Thereafter, BLQ values between evaluable concentrations were substituted by LLOQ/2. LLOQ=1.5 ng/mL.

Following administration of 3 mg/kg and 10 mg/kg 2-hour infusions of PRV-3279, mean peak concentrations occurred at the end of infusion (2 hours) for Days 1, 15, and 29. Mean concentrations were above the lower limit of quantification (LLOQ; 1.5 ng/mL) through 1344 hours following Day 29 administration for both dose levels. Mean pre-dose concentrations on Days 15, 29, and 43 were 6145 ng/mL, 7590 ng/mL, and 12440 ng/mL, respectively, for the 3 mg/kg dose, and 48383 ng/mL, 60460 ng/mL, and 77140 ng/mL, respectively, for the 10 mg/kg dose. As the pre-dose concentrations on these days are continuing to increase and less than 5 half-lives had elapsed, steady-state was not achieved on Day 15 nor on Day 29.

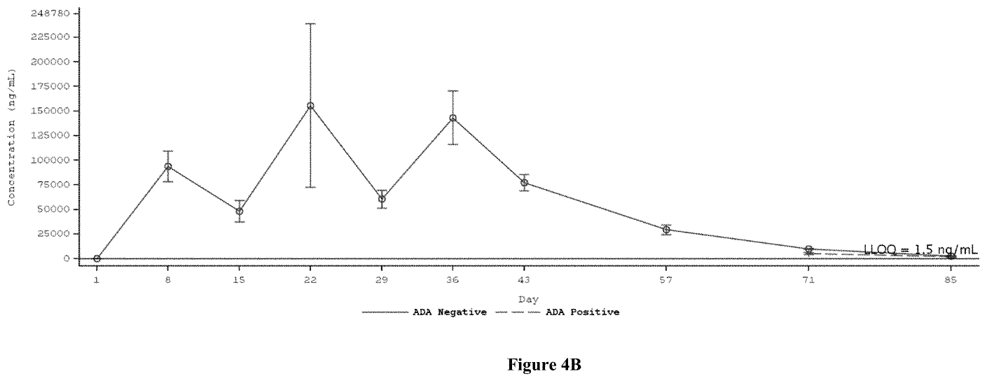

The arithmetic mean PRV-3279 concentration-time data are displayed by treatment and ADA results in FIGS. 4A-4B. The ADA results in this plot are defined based on immunogenicity sample results at each specific time point. ADA=antidrug antibody; BLQ=below the limit of quantification; LLOQ; lower limit of quantification; SD=standard deviation. Values that were BLQ at pre-dose and in the absorption phase before the first quantifiable concentration were substituted by zeros. Thereafter, BLQ values between evaluable concentrations were substituted by LLOQ/2. LLOQ=1.5 ng/mL. For 3 mg/kg, ADA Negative/Positive by Day: Day 1 and 8=6/0 (N=6); Day 15, 22 and 29=5/1 (N=6); Day 36=4/2 (N=6); Day 43=3/3 (N=6); Day 57=2/4 (N=6); Day 71=1/5 (N=6); Day 85 0/6 (N=6). For 10 mg/kg, ADA Negative/Positive by Day: Day 1, 8, 15, 22=6/0 (N=6); Day 29=5/0 (N=5); Day 36=5/0 (N=5); Day 43=5/0 (N=5); Day 57=5/0 (N=5); Day 71=3/2 (N=5); Day 85 2/3 (N=5).

### Immunogenicity Data Evaluations

Anti-PRV-3279 antibodies in human serum are detected and confirmed in human serum using a multi-tiered approach in an MSD-ECL assay. In this assay, samples, positive controls (PCs), and negative control (NC) are subjected to a 1:10 minimum required dilution (MRD) in 300 mM acetic acid. The acidified samples are then neutralized and pre-incubated overnight with Biotin-PRV-3279 coated on a NeutrAvidin high capacity plate. Any antidrug antibodies (ADA) present in the human serum will bind to Biotin-PRV-3279. After an overnight incubation, Biotin-PRV-3279:ADA complexes are subjected to a second acid treatment to break the complexes. Acidified ADA samples are then coated on bare MSD high bind plate. After blocking, ADA samples are detected with Sulfo-Tag-PRV-3279 by a chemiluminescent signal that is generated when voltage is applied. The resulting electrochemiluminescent (ECL) signal, or relative light units (RLU), is directly proportional to the amount of ADA present in the human serum.

Overall, ADAs increased over time. Antidrug antibodies in subjects on the 3 mg/kg dose developed ADAs earlier (Day 15 versus Day 36) and all subjects developed ADAs by Day 85 compared to subjects on the 10 mg/kg dose who only had 4 out of 6 subjects who had ADAs at Day 85. Titers of ADAs to PRV-3279 from baseline over time are shown in Table 7 and ranged from <10 to 270 and <10 to 2430. This shows that PRV-3279 inhibits its own immunogenicity.

TABLE 7
Incidence of ADA Positive Results by Titer, Treatment and Overall
(Immunogenicity Population)

| | | | | | | | Pooled | | |
|---|---|---|---|---|---|---|---|---|---|
| | 3 mg/kg PRV-3279 | | | 10 mg/kg PRV-3279 | | | Placebo | Total | |
| | Titer for | (N = 6) | | Titer for | (N = 6) | | (N = 4) | (N = 16) | |
| | ADA | n (%) | | ADA | n (%) | | n (%) | n (%) | |
| Visit | Positive | n/n' (%) | | Positive | n/n' (%) | | n/n' (%) | n/n' (%) | |
| Day 15 | 30 | 1/1 | (100.0) | — | 0 | | 0 | 1/1 | |
| Day 22 | 90 | 1/1 | (100.0) | — | 0 | | 0 | 1/1 | |
| Day 29 | 270 | 1/1 | (100.0) | — | 0 | | 0 | 1/1 | (100.0) |
| Day 36 | 10-90 | 2/2 | (100.0) | 30 | 1/1 | (100.0) | 0 | 3/3 | (100.0) |
| Day 43 | <10-30 | 3/3 | (100.0) | 90 | 1/1 | (100.0) | 0 | 4/4 | (100.0) |
| Day 57 | <10-30 | 4/4 | (100.0) | 810 | 1/1 | (100.0) | 0 | 5/5 | (100.0) |
| Day 71 | 30-270 | 5/5 | (100.0) | <10-2430 | 3/3 | (100.0) | 0 | 8/8 | (100.0) |
| Day 85 | <10-270 | 6/6 | (100.0) | <10-2430 | 4/4 | (100.0) | 0 | 10/10 | (100.0) |

ADA = antidrug antibody, N = number of subjects in the analysis population; n = number of subjects within a category
For ADA positive, n' represented the number of subjects with available ADA results at each timepoint.
For titer, n' represented the number of subjects with ADA positive at each timepoint.
Percentages were based on n'.

### Pharmacokinetic/Immunogenicity Data Evaluations

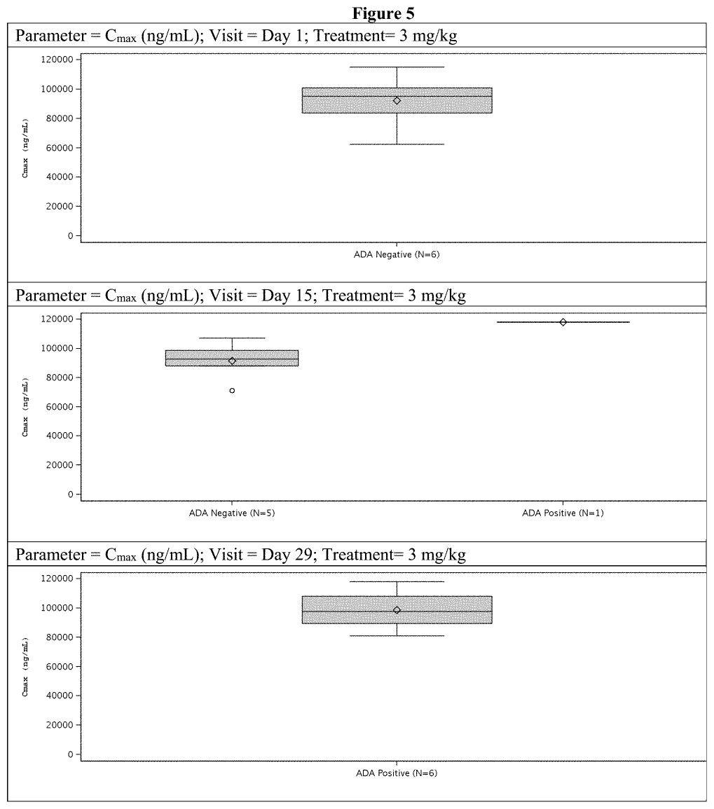

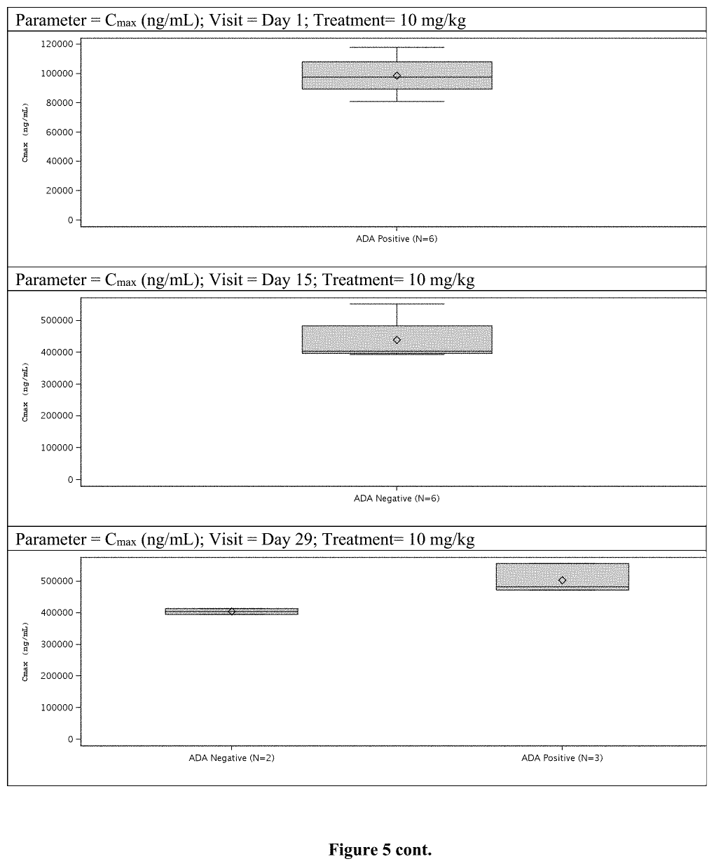

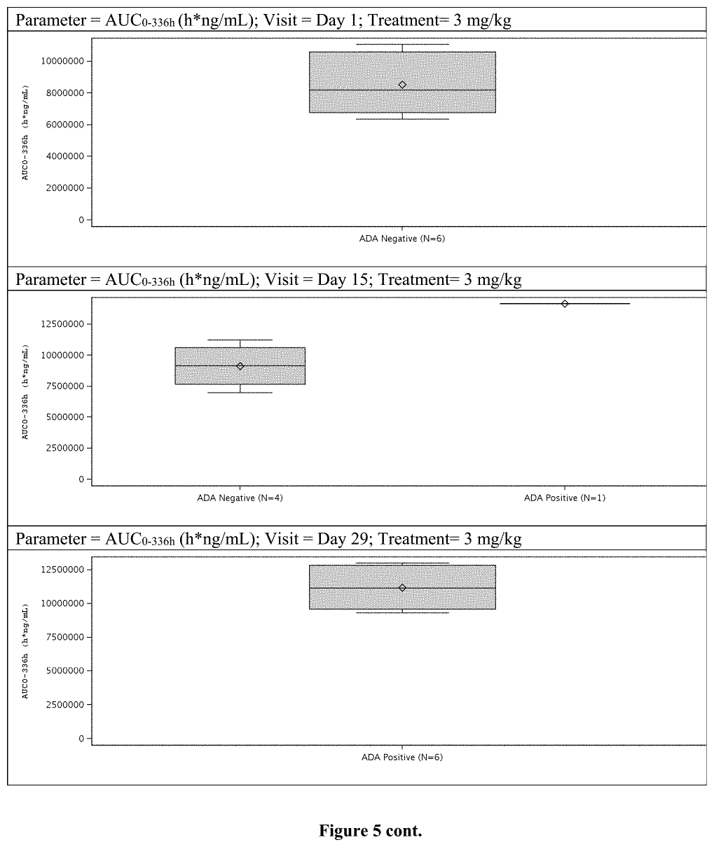

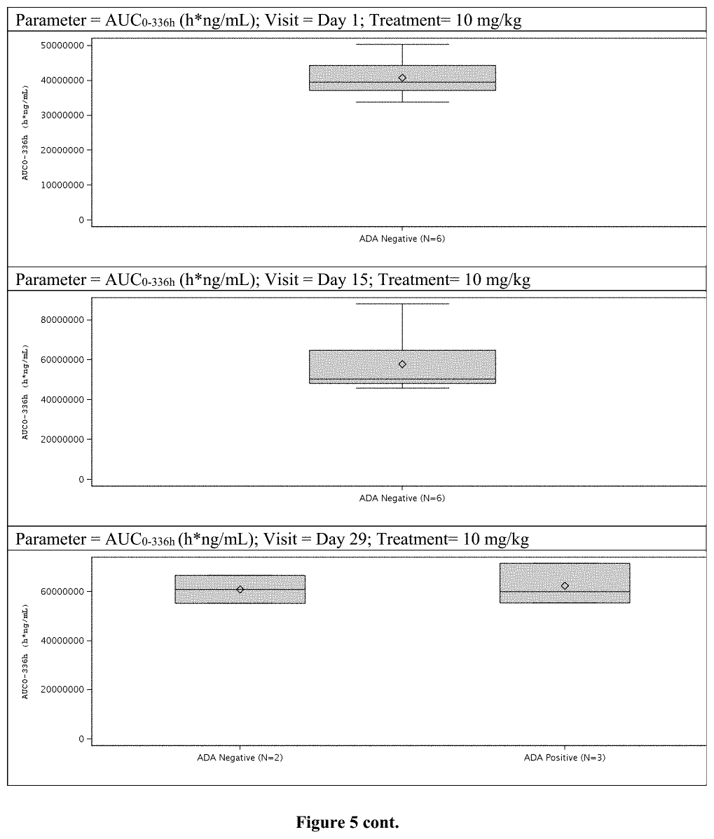

Pharmacokinetic parameters (Cₘₐₓ and AUC₀₋₃₃₆) for PRV-3279 are summarized descriptively by ADA result, treatment, and day in Table 8. Box plots of PRV-3279 Cₘₐₓ and AUC₀₋₃₃₆ parameters are presented in FIG. 5, showing that ADA does not affect PK. ADA=antidrug antibody; N=number of subjects in pharmacokinetic population in respective ADA category. The symbol in the box interior represents the mean. The upper (lower) edge of the box represents the 75th (25th) percentile. A whisker is drawn from the upper (lower) edge of the box to the largest (smallest) value within 1.5× interquartile range above (below) the edge of the box. Values outside the whiskers are identified with symbols.

TABLE 8
Summary of PRV-3279 Serum Pharmacokinetic Parameters by Treatment and Day
(Pharmacokinetic Population)

| | | PRV-3279 | | | PRV-3279 | | |
|---|---|---|---|---|---|---|---|
| | | 3 mg/kg | | | 10 mg/kg | | |
| Parameter | | (N = 6) | | | (N = 6) | | |
| (Unit) | Statistics | Day 1 | Day 15 | Day 29 | Day 1 | Day 15 | Day 29* |
| Cₘₐₓ (ng/mL) | n | 6 | 6 | 6 | 6 | 6 | 5 |
| | Mean | 92100 | 95900 | 98700 | 362000 | 439000 | 464000 |
| | SD | 17700 | 16200 | 14100 | 118000 | 65000 | 63500 |
| | CV (%) | 19.2 | 16.9 | 14.2 | 32.6 | 14.8 | 13.7 |
| | Geo Mean | 90600 | 94700 | 97900 | 346000 | 435000 | 461000 |
| | Geo CV (%) | 21.0 | 17.6 | 14.4 | 34.7 | 14.0 | 13.6 |
| Tₘₐₓ (h) | Median | 2.01 | 2.00 | 2.01 | 2.00 | 2.00 | 2.00 |
| | Minimum | 2.00 | 2.00 | 2.00 | 2.00 | 2.00 | 2.00 |
| | Maximum | 2.12 | 24.03 | 2.05 | 24.07 | 2.02 | 2.02 |
| AUC₀₋ₜ | n | 6 | 6 | 6 | 6 | 6 | 5 |
| (h*ng/mL) | Mean | 8550000 | 10300000 | 13700000 | 41000000 | 58100000 | 84800000 |
| | SD | 2020000 | 2510000 | 2880000 | 5930000 | 16100000 | 6650000 |
| | CV (%) | 23.6 | 24.5 | 21.0 | 14.5 | 27.7 | 7.8 |
| | Geo Mean | 8360000 | 10000000 | 13500000 | 40600000 | 56600000 | 84600000 |
| | Geo CV (%) | 23.9 | 25.0 | 20.6 | 14.3 | 25.2 | 7.8 |
| AUC₀₋₃₃₆ | n | 6 | 5 | 6 | 6 | 6 | 5 |
| (h*ng/mL) | Mean | 8530000 | 10100000 | 11200000 | 40800000 | 58000000 | 61800000 |
| | SD | 2000000 | 2770000 | 1580000 | 5910000 | 16200000 | 7160000 |
| | CV (%) | 23.4 | 27.2 | 14.1 | 14.5 | 28.0 | 11.6 |
| | Geo Mean | 8340000 | 9850000 | 11100000 | 40500000 | 56400000 | 61500000 |
| | Geo CV (%) | 23.7 | 27.6 | 14.3 | 14.2 | 25.4 | 11.5 |
| AUC_{0-∞} | n | 6 | 4 | 6 | 4 | 3 | 5 |
| (h*ng/mL) | Mean | 9470000 | 12200000 | 13700000 | 50900000 | 61600000 | 85400000 |
| | SD | 2620000 | 3200000 | 2910000 | 6370000 | 11800000 | 6540000 |
| | CV (%) | 27.7 | 26.1 | 21.2 | 12.5 | 19.2 | 7.7 |
| | Geo Mean | 9180000 | 11900000 | 13500000 | 50600000 | 60900000 | 85200000 |
| | Geo CV (%) | 27.5 | 26.0 | 20.7 | 13.1 | 18.5 | 7.6 |
| T_{1/2} (h) | n | 6 | 4 | 6 | 4 | 3 | 5 |
| | Mean | 94.1 | 99.9 | 160 | 127 | 114 | 186 |
| | SD | 19.9 | 16.6 | 30.4 | 16.4 | 2.25 | 24.2 |
| | CV (%) | 21.1 | 16.6 | 19.1 | 12.9 | 2.0 | 13.0 |
| | Geo Mean | 92.5 | 98.9 | 157 | 126 | 114 | 185 |
| | Geo CV (%) | 19.5 | 16.6 | 18.6 | 13.8 | 2.0 | 13.3 |
| CL | n | NC | NC | 6 | NC | NC | 5 |
| (mL/h/kg) | Mean | NC | NC | 2.73 | NC | NC | 1.63 |
| | SD | NC | NC | 0.389 | NC | NC | 0.183 |
| | CV (%) | NC | NC | 14.2 | NC | NC | 11.2 |
| | Geo Mean | NC | NC | 2.71 | NC | NC | 1.63 |
| | Geo CV (%) | NC | NC | 14.3 | NC | NC | 11.5 |
| Vₛₛ (mL/kg) | n | NC | NC | 6 | NC | NC | 5 |
| | Mean | NC | NC | 632 | NC | NC | 591 |
| | SD | NC | NC | 141 | NC | NC | 136 |
| | CV (%) | NC | NC | 22.4 | NC | NC | 23.0 |
| | Geo Mean | NC | NC | 618 | NC | NC | 576 |
| | Geo CV (%) | NC | NC | 23.6 | NC | NC | 26.8 |
| MRT (h) | n | NC | NC | 6 | NC | NC | 5 |
| | Mean | NC | NC | 237 | NC | NC | 358 |
| | SD | NC | NC | 73.7 | NC | NC | 52.3 |
| | CV (%) | NC | NC | 31.1 | NC | NC | 14.6 |
| | Geo Mean | NC | NC | 228 | NC | NC | 354 |
| | Geo CV (%) | NC | NC | 30.4 | NC | NC | 16.4 |
| C_{trough} | n | NC | 6 | 6 | NC | 6 | 4 |
| (ng/mL) | Mean | NC | 7590 | 12400 | NC | 57400 | 80500 |
| | SD | NC | 3080 | 4730 | NC | 10900 | 4110 |
| | CV (%) | NC | 40.6 | 38.1 | NC | 19.1 | 5.1 |
| | Geo Mean | NC | 7140 | 11700 | NC | 56400 | 80400 |
| | Geo CV (%) | NC | 38.4 | 37.9 | NC | 20.6 | 5.1 |
| RacCₘₐₓ | n | NC | NC | 6 | NC | NC | 5 |
| | Mean | NC | NC | 1.09 | NC | NC | 1.32 |
| | SD | NC | NC | 0.179 | NC | NC | 0.511 |
| | CV (%) | NC | NC | 16.4 | NC | NC | 38.8 |
| | Geo Mean | NC | NC | 1.08 | NC | NC | 1.25 |
| | Geo CV (%) | NC | NC | 15.3 | NC | NC | 33.2 |
| RacAUC₀₋₃₃₆ | n | NC | NC | 6 | NC | NC | 5 |
| | Mean | NC | NC | 1.34 | NC | NC | 1.50 |
| | SD | NC | NC | 0.167 | NC | NC | 0.0824 |
| | CV (%) | NC | NC | 12.5 | NC | NC | 5.5 |
| | Geo Mean | NC | NC | 1.33 | NC | NC | 1.49 |
| | Geo CV (%) | NC | NC | 13.2 | NC | NC | 5.4 |

CV = coefficient of variation; Geo = geometric; N = number of subjects in pharmacokinetic population in respective treatment; n = number of subjects in respective category; NC = not calculated; SD = standard deviation
*Subject/Randomization No. 113/55 (Cohort B, 10 mg/kg) not included

### Pharmacodynamic Data Evaluations

PRV-3279 binding (percent B cells bound) and absolute and percent receptor occupancy (MESF) by staining of anti-PRV-3279 (anti-EK) on B cells (CD19+), memory B cells (CD19+/CD27+) and naïve B cells (CD19+/CD27-) are examined, including the maximum binding obtained in a PRV-3279 saturated sample. For % B cells bound and % receptor occupancy calculations, the maximum binding of PRV-3279 to B cells in each individual sample was calculated by comparing the % cells bound by anti-PRV-3279 (anti-EK) and the absolute receptor occupancy molecules of equivalent soluble fluorochromes (MESF) values at each time point to the respective values of a PRV-3279 saturated sample (total).

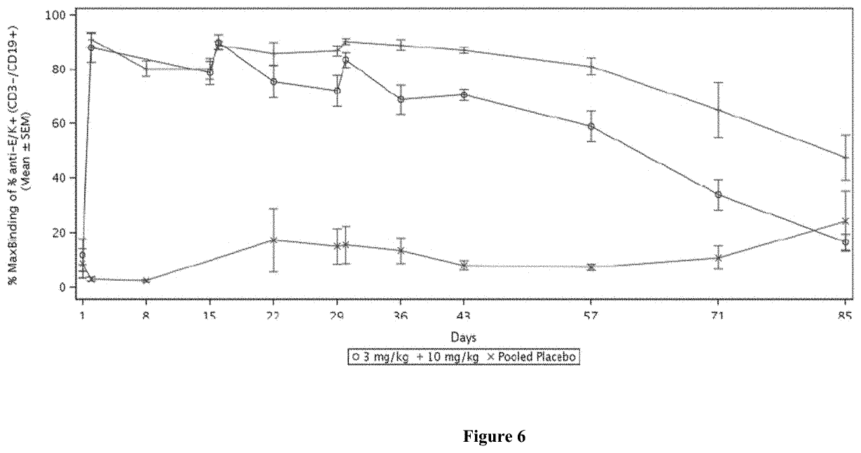

As shown in FIG. 6, following a dose of 3 and 10 mg/kg PRV-3279>85% of total available B cells (CD19+) were bound by the drug 1 day after dosing in both dose groups. Binding slightly decreased to about 80% before the second dose and was increased again to about 90% following the second dose in both dose groups. In the 10 mg/kg dose group, % B cells bound was maintained on this high level until approximately Day 57 before it declined to below 50% on Day 85 (see FIG. 6). For the 3 mg/kg dose, % B cells bound was comparable to the higher dose for first 22 days and remained around 70% until Day 43 but binding generally fluctuated more at this dose level between administrations. Percent B cells bound was <20% on Day 85, which was in the same range as with placebo. The variability of data was low to moderate.

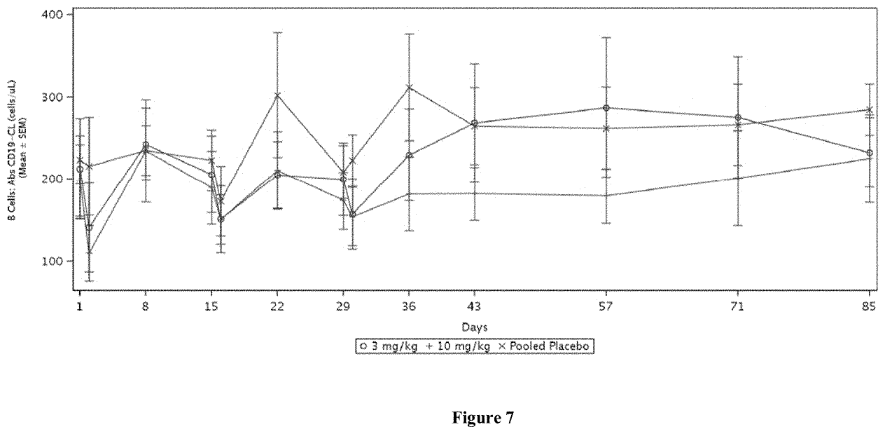

Next, percentage and absolute numbers of lymphocytes, monocytes (CD14+), T cells (CD3+), T-helper cells (CD3+/CD4+), cytotoxic T cells (CD3+/CD8+), Natural Killer Cells (CD3-/CD16+), Natural Killer T Cells (CD3+/CD16+/CD56+) and B cells (CD19+) were investigated. The time course of absolute number of B cells is shown by treatment in FIG. 7. The other cell types show similar pattern (data not shown). The number of B cells dropped by −39% and −47% on average after dosing of 3 and 10 mg/kg PRV-3279, respectively, within 1 day but returned to baseline levels after 1 week. No comparable reduction in placebo treated subjects was observed. The fall in cell numbers was slightly less pronounced after the second and third dose of PRV-3279. Following the third dose, cell counts remained lower for 10 mg/kg compared to 3 mg/kg but both counts were similar on Day 85 and comparable to baseline again.

In summary, the study demonstrated an initial, transient decrease in peripheral B cell counts, which was less pronounced after second and third dose of PRV-3279 and quickly recovered after each dose. No sustained depletion of B cells occurred in this study. Also, none of the other immune cell types investigated showed clinically relevant depletion.

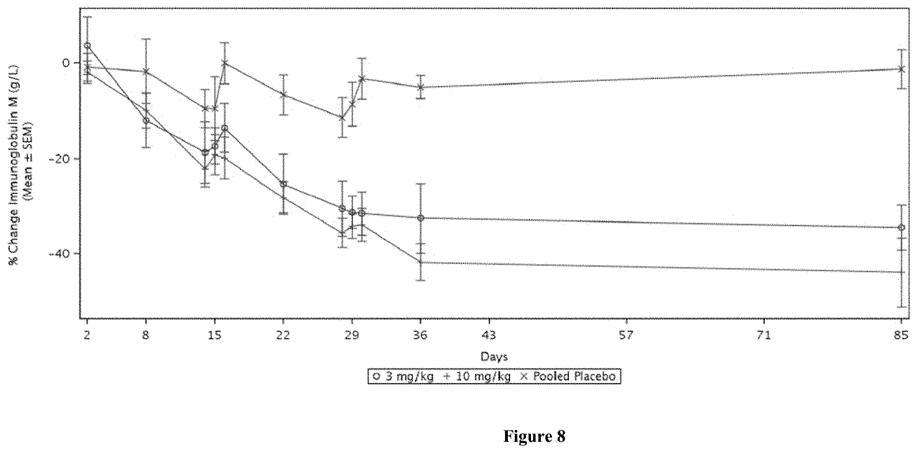

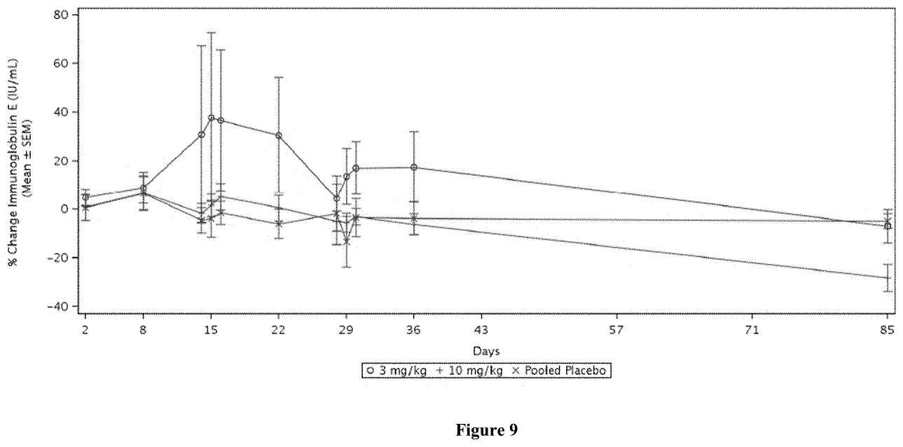

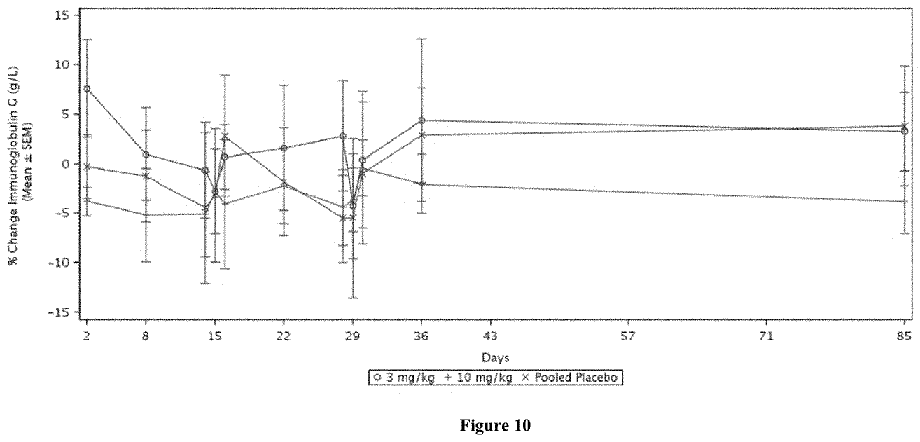

Next, the circulating levels of immunoglobulin M (IgM), IgE and IgG are measured using known methods. As shown in FIG. 8, immunoglobulin M levels after dosing of PRV-3279 of 3 and 10 mg/kg steadily decreased to approximately Day 36 and remained on that level until Day 85. Decreases were not clearly dose-dependent, however, trended to be less pronounced for 3 mg/kg compared to 10 mg/kg on Days 36 and 85. Immunoglobulin E levels after dosing of PRV-3279 of 10 mg/kg were similar to the values observed for placebo over the course of the study except for the last time point on Day 85, where the average % change from baseline was −28.2% for 10 mg/kg compared to −5.0% for placebo (see FIG. 9). Immunoglobulin G levels after dosing of PRV-3279 of 3 and 10 mg/kg were highly variable and generally appeared not to be different to placebo over the course of the study. % Changes from baseline in IgG levels were mostly within ±5% for all treatments (see FIG. 10).

### CONCLUSIONS

The primary objective of this Phase 1b, double-blind, placebo-controlled, MAD study was to assess the safety and tolerability of multiple (3) IV infusions of 2 dose levels of PRV-3279 (3 and 10 mg/kg) in healthy subjects. The secondary objectives were to characterize the multidose PK and the immunogenicity of PRV-3279. The exploratory objective was to explore the effects of PRV-3279 on potential biomarkers for target engagement and B cell function.

A total of 16 subjects were enrolled, randomized, and dosed. Two cohorts were administered PRV-3279 or placebo every 2 weeks for a total of 3 doses. The 3 doses of study drug (PRV-3279 3 mg/kg and 10 mg/kg or placebo) were administered IV on Day 1, Day 15, and Day 29 in each cohort. Fourteen subjects received all planned treatments per protocol and completed the study. One placebo subject withdrew consent after receiving the Day 1 and Day 15 placebo administrations and 1 PRV-3279 10 mg/kg subject was withdrawn due to AEs after receiving 3 minutes of the Day 29 PRV-3279 10 mg/kg administration.

All 16 (100.0%) subjects were included in the safety, PD, and immunogenicity populations. All 12 (75.5%) subjects who received study drug were included in the PK population; however, for all PK summary plots and summary statistics, data from Day 29 onward were excluded for the 1 PRV-3279 10 mg/kg subject who was withdrawn due to AEs.

This Phase 1b study builds on the tolerability and PD information obtained in the First-in-Human study and addresses the feasibility of re-dosing PRV-3279. The results of the study confirm the ability of PRV-3279 to functionally suppress, without depleting, B cell function, in a profound and durable fashion which is not affected by ADA.

PRV-3279 was well-tolerated, with no SAES. The PK characteristics support bi-weekly or possibly less frequent dosing. Antidrug antibodies were lower in the higher dose group, consistent with the ability of PRV-3279 to inhibit its own immunogenicity.

The receptor occupancy PD effect of PRV-3279 persisted well beyond the cessation of dosing, with greater persistence with the 10 mg/kg dose and >50% binding observed at least 28 days after the final dose, which is considered the minimum level of binding required for optimal B cell modulation.

There was clear reduction in IgM levels that persisted through the follow-up period, suggesting an extended PD effect. Importantly, and as expected, there was no B cell depletion nor any observable detrimental effects on immune cells or cytokines.

In conclusion and based upon the excellent safety profile and superior PD effects at 10 mg/kg with lower immunogenicity, the 10 mg/kg or higher dose can be used for the reduction of the immunogenicity of biotherapeutics including gene therapy products.

### MODIFICATIONS

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A method of reducing immunogenicity, comprising administering to a patient receiving or having received a biological therapeutic agent, an effective amount of B cell inhibitor that is non-depletional.

2. The method of claim 1, wherein the biological therapeutic agent is selected from one or more of: gene therapy, gene editing therapy, messenger RNA (mRNA) therapy, oncolytic viruses, enzyme replacement therapy, antibody therapy, protein therapeutics, and cell therapy.

3. The method of claim 1, wherein the biological therapeutic agent is gene therapy.

4. The method of claim 1, wherein the B cell inhibitor is a CD32B×CD79B bi-specific antibody capable of immunospecifically binding an epitope of CD32B and an epitope of CD79B.

5. The method of claim 4, wherein the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

6. The method of claim 4, wherein said CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

7. The method of claim 6, comprising administering the Fc diabody at a dose of between about 5 mg/kg and about 40 mg/kg, and at a dosage regimen of between one dose per 2 week and one dose per 6 weeks.

8. The method of claim 6, comprising administering the Fc diabody at a dose of about 10 mg/kg, and at a dosage regimen of one dose per 4 weeks.

9. The method of claim 6, comprising administering 3 doses of the Fc diabody at a dose of about 10 mg/kg at 2-6 week intervals.

10. The method of claim 9, comprising administering a first dose about 2-6 weeks prior to administration of the biological therapeutic agent, a second dose at about the same time as administration of the biological therapeutic agent, and a third dose about 2-6 weeks after administration of the biological therapeutic agent.

11. The method of claim 6, wherein the Fc diabody results in inhibition of its own immunogenicity upon administration, with lower prevalence and/or titers of anti-drug antibodies (ADA) at increased doses.

12. The method of claim 11, wherein the ADA does not neutralize the Fc diabody.

13. The method of claim 6, wherein the Fc diabody, in a dose-dependent fashion, binds to at least 80% B cells upon administration, and remains bound to at least 50% of the B cells for at least 4 weeks after last administration.

14. The method of claim 6, wherein the Fc diabody results in sustained inhibition of immunoglobulin production without depleting circulating B cells.

15. The method of claim 14, wherein the immunoglobulin includes one or more of IgM, IgA, IgG and IgE.

16. The method of claim 1, further comprising monitoring the patient by examining the presence of specific antibodies against the biological therapeutic agent.

17. The method of claim 16, further comprising administering one or more dose of the B cell inhibitor to further modulate immunogenicity.

18. The method of claim 1, further comprising co-administering one or more immune-modulators.

19. The method of claim 18, wherein the one or more immune-modulators are selected from sirolimus, rapamycin, abatacept, teplizumab and immunoglobulin G-degrading enzyme of Streptococcus pyogenes.
